SEQUENCE LISTING (43 sequences)

SEQ ID NO: 1 - DNA, 1481 bp, Acidovorax avenae subsp. citrulli
attgaacgctggcggcatgccttacacatgcaagtcgaacggtaacaggtcttcggatgctgacgagtggcgaacgggtgagtaatacatcggaacgtgcccgatcgtgggggataacgaggcgaaagctttgctaataccgcataagatctatggatgaaagcaggggaccgtaaggccttgcgcgaacggagcggccgatggcagattaggtagttggtggggtaaaggcttaccaagcctacgatctgtagctggtctgagaggacgaccagccacactgggactgagacacggcccagactcctacgggaggcagcagtggggaattttggacaatgggcgcaagcctgatccagccatgccgcgtgcaggatgaaggccttcgggttgtaaactgcttttgtacggaacgaaaagccttcttctaataaaggggggtcatgacggtaccgtaagaataagcaccggctaactacgtgccagcagccgcggtaatacgtagggtgcaagcgttaatcggaattactgggcgtaaagcgtgcgcaggcggtgatgtaagacagatgtgaaatccccgggctcaacctgggaactgcatttgtgactgcatcgctggagtacggcagagggggatggaattccgcgtgtagcagtgaaatgcgtagatatgcggaggaacaccgatggcgaaggcaatcccctgggcctgtactgacgctcatgcacgaaagcgtggggagcaaacaggattagataccctggtagtccacgccctaaacgatgtcaactggttgttgggtcttcactgactcagtaacgaagctaacgcgtgaagttgaccgcctggggagtacggccgcaaggttgaaactcaaaggaattgacggggacccgcacaagcggtggatgatgtggtttaattcgatgcaacgcgaaaaaccttacccacctttgacatgtacggaatcctttagagatagaggagtgctcgaaagagaaccgtaacacaggtgctgcatggctgtcgtcagctcgtgtcgtgagatgttgggttaagtcccgcaacgagcgcaacccttgccattagttgctacgaaagggcactctaatgggactgccggtgacaaaccggaggaaggtggggatgacgtcaagtcctcatggcccttataggtggggctacacacgtcatacaatggctggtacagagggttgccaacccgcgagggggagctaatcccataaagccagtcgtagtccggatcgcagtctgcaactcgactgcgtgaagtcggaatcgctagtaatcgcggatcagaatgtcgcggtgaatacgttcccgggtcttgtacacaccgcccgtcacaccatgggagcgggttctgccagaagtaggtagcctaaccgtaaggagggcgcttaccacggcagggttcgtgactggggtgaagtcgtaacaaggtacccgtatcggaagg

SEQ ID NO: 2 - DNA, 18 bp, artificial sequence
  primer
aagtcgtaacaaggtarc

SEQ ID NO: 3 - DNA, 18 bp, artificial sequence
  primer
aagtcgtaacaaggtacc

SEQ ID NO: 4 - DNA, 15 bp, artificial sequence
  primer BITS-2
ggttbccccattcrg

SEQ ID NO: 5 - DNA, 711 bp, Acidovorax avenae subsp. citrulli
gcggctggatcacctcctttctggaaaacagcattcaatattgaacgcccacacttatcggttgttggaagaagtcggtgctaaccgacatgggtctgtagctcagctggttagagcaccgtcttgataaggcgggggtcgttggttcgagcccaactagacccaccaaatcttccgaacataagatgcgaggatcagkgggggattagctcagctgggagagcacctgctttgcaagcagggggtcgtcggttcgatcccgtcatcctccaccaaccaatacgctctgcggtagggcgaagaaaccaacaccaaagcggcttcgcgagaggcctctttgttgttggtccggtatagaccggatcaatcggctgttctttaaaaattcatagagtcgaatcagcgttgccggcggaaagcaggaaactgcaccgtgccgccggtgacaaaaatttgattgcgtcaaaacgaatattcaattgagcgaaagcttgttgaaattcagtaatgacgaattgttctctaggtagcaataccgaagaagaattcacattacggcataacgcgcgaggtgaaagacctcgcaagtccttgaaagaaagcggagatgtctcgcaagagatgtcaaagttatagggtcaagtgactaagagcatgtggtggatgccttggcgatgataggcgacgaaagacgtgatagcctgcgataag

SEQ ID NO: 6 - DNA, 20 bp, artificial sequence
  primer Aac-BITS3
ctttcacctcgcgcgttatg

SEQ ID NO: 7 - DNA, 20 bp, artificial sequence
  primer Aac-BITS4
tgaacgcccacacttatcgg

SEQ ID NO: 8 - DNA, 20 bp, artificial sequence
  primer Aac-BITS6
ccgataagtgtgggcgttca

SEQ ID NO: 9 - DNA, 20 bp, artificial sequence
  primer Aac-BITS5
tcgccaaggcatccaccaca

SEQ ID NO: 10 - DNA, 22 bp, artificial sequence
  primer Aac-BITS7
tcgtagtccggatcgcagtctg

SEQ ID NO: 11 - DNA, 15 bp, artificial sequence
  primer 1100F
caacgagcgcaaccc

SEQ ID NO: 12 - DNA, 478 bp, Acidovorax avenae subsp. citrulli
caacgagcgcaacccttgccattagttgctacgaaagggcactctaatgggactgccggtgacaaaccggaggaaggtggggatgacgtcaagtcctcatggcccttataggtggggctacacacgtcatacaatggctggtacagagggttgccaacccgcgagggggagctaatcccataaagccagtcgtagtccggatcgcagtctgcaactcgactgcgtgaagtcggaatcgctagtaatcgcggatcagaatgtcgcggtgaatacgttcccgggtcttgtacacaccgcccgtcacaccatgggagcgggttctgccagaagtaggtagcctaaccgtaaggagggcgcttaccacggcagggttcgtgactggggtgaagtcgtaacaaggtagccgtatcggaaggtgcggctggatcacctcctttctggaaaacagcattcaatattgaacgcccacacttatcgg

SEQ ID NO: 13 - DNA, 480 bp, Acidovorax avenae subsp. citrulli
caacgagcgcaacccttgccattagttgctacgaaagggcactctaatgggactgccggtgacaaaccggaggaaggtggggatgacgtcaagtcctcatggcccttataggtggggctacacacgtcatacaatggctggtacagagggttgccaacccgcgagggggagctaatcccataaagccagtcgtagtccggatcgcagtctgcaactcgactgcgtgaagtcggaatcgctagtaatcgcggatcagaatgtcgcggtgaatacgttcccgggtcttgtacacaccgcccgtcacaccatgggagcgggttctgccagaagtaggtagcctaaccgtaaggagggcgcttaccacggcagggttcgtgactggggtgaagtcgtaacaaggtagccgtatcggaaggtgcggctggatcacctcctttctggaaaacagcattcaatattgaacgcccacacttatcaagg

SEQ ID NO: 14 - DNA, 993 bp, Acidovorax avenae subsp. citrulli
tcgtagtccggatcgcagtctgcaactcgactgcgtgaagtcggaatcgctagtaatcgcggatcagaatgtcgcggtgaatacgttcccgggtcttgtacacaccgcccgtcacaccatgggagcgggttctgccagaagtaggtagcctaaccgtaaggagggcgcttaccacggcagggttcgtgactggggtgaagtcgtaacaaggtagccgtatcggaaggtgcggctggatcacctcctttctggaaaacagcattcaatattgaacgcccacacttatcggttgttggaagaagtcggtgctaaccgacatgggtctgtagctcagctggttagagcaccgtcttgataaggcgggggtcgttggttcgagcccaactagacccaccaaatcttccgaacataagatgcgaggatcagtgggggattagctcagctgggagagcacctgctttgcaagcagggggtcgtcggttcgatcccgtcatcctccaccaaccaatacgctctgcggtagggcgaagaaaccaacaccaaagcggcttcgcgagaggcctctttgttgttggtccggtatagaccggatcaatcggctgttctttaaaaattcatagagtcgaatcagcgttgccggcggaaagcaggaaactgcaccgtgccgccggtgacaaaaatttgattgcgtcaaaacgaatattcaattgagcgaaagcttgttgaaattcagtaatgacgaattgttctctaggtagcaataccgaagaagaattcacattacggcataacgcgcgaggtgaaagacctcgcaagtccttgaaagaaagcggagatgtctcgcaagagatgtcaaagttatagggtcaagtgactaagagcatgtggtggatgccttggcgatgataggcgacgaaagacgtgatagcctgcgataagcttcggggagctggcaaataagctttgatccggagatttctgaatggggcaacc

SEQ ID NO: 15 - DNA, 993 bp, Acidovorax avenae subsp. citrulli
tcgtagtccggatcgcagtctgcaactcgactgcgtgaagtcggaatcgctagtaatcgcggatcagaatgtcgcggtgaatacgttcccgggtcttgtacacaccgcccgtcacaccatgggagcgggttctgccagaagtaggtagcctaaccgtaaggagggcgcttaccacggcagggttcgtgactggggtgaagtcgtaacaaggtagccgtatcggaaggtgcggctggatcacctcctttctggaaaacagcattcaatattgaacgcccacacttatcggttgttggaagaagtcggtgctaaccgacatgggtctgtagctcagctggttagagcaccgtcttgataaggcgggggtcgttggttcgagcccaactagacccaccaaatcttccgaacataagatgcgaggatcagtgggggattagctcagctgggagagcacctgctttgcaagcagggggtcgtcggttcgatcccgtcatcctccaccaaccaatacgctctgcggtagggcgaagaaaccaacaccaaagcggcttcgcgagaggcctctttgttgttggtccggtatagaccggatcaatcgactgttctttaaaaattcatagagtcgaatcagcgttgccggcggaaagcaggaaactgcaccgtgccgccggtgacaaaaatttgattgcgtcaaaacgaatattcaattgagcgaaagcttgttgaaattcagtaatgacgaattgttctctaggtagcaataccgaagaagaattcacattacggcataacgcgcgaggtgaaagacctcgcaagtccttgaaagaaagcggagatgtctcgcaagagatgtcaaagttatagggtcaagtgactaagagcatgtggtggatgccttggcgatgataggcgacgaaagacgtgatagcctgcgataagcttcggggagctggcaaataagctttgatccggagatttctgaatggggcaacc

SEQ ID NO: 16 - DNA, 993 bp, Acidovorax avenae subsp. citrulli
tcgtagtccggatcgcagtctgcaactcgactgcgtgaagtcggaatcgctagtaatcgcggatcagaatgtcgcggtgaatacgttcccgggtcttgtacacaccgcccgtcacaccatgggagcgggttctgccagaagtaggtagcctaaccgtaaggagggcgcttaccacggcagggttcgtgactggggtgaagtcgtaacaaggtagccgtatcggaaggtgcggctggatcacctcctttctggaaaacagcattcaatattgaacgcccacacttatcggttgttggaagaagtcggtgctaaccgacatgggtctgtagctcagctggttagagcaccgtcttgataaggcgggggtcgttggttcgagcccaactagacccaccaaatcttccgaacataagatgcgaggatcagtgggggattagctcagctgggagagcacctgctttgcaagcagggggtcgtcggttcgatcccgtcatcctccaccaaccaatacgctctgcggtagggcgaagaaaccaacaccaaagcggcttcgcgagaggcctctttgttgttggtccggtatagaccggatcaatcgrctgttctttaaaaattcatagagtcgaatcagcgttgccggcggaaagcaggaaactgcaccgtgccgccggtgacaaaaatttgattgcgtcaaaacgaatattcaattgagcgaaagcttgttgaaattcagtaatgacgaattgttctctaggtagcaataccgaagaagaattcacattacggcataacgcgcgaggtgaaagacctcgcaagtccttgaaagaaagcggagatgtctcgcaagagatgtcaaagttatagggtcaagtgactaagagcatgtggtggatgccttggcgatgataggcgacgaaagacgtgatagcctgcgataagcttcggggagctggcaaataagctttgatccggagatttctgaatggggcaacc

SEQ ID NO: 17 - DNA, 993 bp, Acidovorax avenae subsp. citrulli; identical to SEQ ID NO: 14

SEQ ID NO: 18 - DNA, 993 bp, Acidovorax avenae subsp. citrulli
tcgtagtccggatcgcagtctgcaactcgactgcgtgaagtcggaatcgctagtaatcgcggatcagaatgtcgcggtgaatacgttcccgggtcttgtacacaccgcccgtcacaccatgggagcgggttctgccagaagtaggtagcctaaccgtaaggagggcgcttaccacggcagggttcgtgactggggtgaagtcgtaacaaggtagccgtatcggaaggtgcggctggatcacctcctttctggaaaacagcattcaatattgaacgcccacacttatcggttgttggaagaagtcggtgctaaccgacatgggtctgtagctcagctggttagagcaccgtcttgataaggcgggggtcgttggttcgagcccaactagacccaccaaatcttccgaacataagatgcgaggatcagtgggggattagctcagctgggagagcacctgctttgcaagcagggggtcgtcggttcgatcccgtcatcctccaccaaccaatacgctctgcggtagggcgaagaaaccaacaccaaagcggcttcgcgagaggcctctttgttgttggtccggtatagaccggatcaatcggctgttctttaaaaattcatagagtcgaatcagcgttgccggcggaaagcaggaaactgcaccgtgccgccggtgacaaaaatttgattgcgtcaaaacgaatattcaattgagcgaaagcttgttgaaattcagtaatgacgaattgttctctaggtagcaataccgaagaagaattcacattacggcataacgcgcgaggtgaaagacctcgcaagtccttgaaagaaagcggagatgtctcgcaagagatgtcaaagttatagggtcaagtgactaagagcatgtggtggatgccttggcgatgataggcgacgaaagacgtgatagcctgcgataagcttcggggagctggcaaataagctttgatccggagatttctgaatggggaaacc

SEQ ID NO: 19 - DNA, 996 bp, Acidovorax avenae subsp. citrulli
tcgcccttgtccggatcgcagtctgcaactcgactgcgtgaagtcggaatcgctagtaatcgcggatcagaatgtcgcggtgaatacgttcccgggtcttgtacacaccgcccgtcacaccatgggagcgggttctgccagaagtaggtagcctaaccgtaaggagggcgcttaccacggcagggttcgtgactggggtgaagtcgtaacaaggtagccgtatcggaaggtgcggctggatcacctcctttctggaaaacagcattcaatattgaacgcccacacttatcggttgttggaagaagtcggtgctaaccgacatgggtctgtagctcagctggttagagcaccgtcttgataaggcgggggtcgttggttcgagcccaactagacccaccaaatcttccgaacataagatgcgaggatcagtgggggattagctcagctgggagagcacctgctttgcaagcagggggtcgtcggttcgatcccgtcatcctccaccaaccaatacgctctgcggtagggcgaagaaaccaacaccaaagcggcttcgcgagaggcctctttgttgttggtccggtatagaccggatcaatcggctgttctttaaaaattcatagagtcgaatcagcgttgccggcggaaagcaggaaactgcaccgtgccgccggtgacaaaaatttgattgcgtcaaaacgaatattcaattgagcgaaagcttgttgaaattcagtaatgacgaattgttctctaggtagcaataccgaagaagaattcacattacggcataacgcgcgaggtgaaagacctcgcaagtccttgaaagaaagcggagatgtctcgcaagagatgtcaaagttatagggtcaagtgactaagagcatgtggtggatgccttggcgatgataggcgacgaaagacgtgatagcctgcgataagcttcggggagctggcaaataagctttgatccggagatttctgaatggggcaacc

SEQ ID NO: 20 - DNA, 993 bp, Acidovorax avenae subsp. citrulli; identical to SEQ ID NO: 18

SEQ ID NO: 21 - DNA, 755 bp, Acidovorax avenae subsp. citrulli
cagtctgcaactcgactgcgtgaagtcggaatcgctagtaatcgcggatcagaatgtcgcggtgaatacgttcccgggtcttgtacacaccgcccgtcacaccatgggagcgggttctgccagaagtaggtagcctaaccgtaaggagggcgcttaccacggcagggttcgtgactggggtgaagtcgtaacaaggtagccgtatcggaaggtgcggctggatcacctcctttctggaaaacagcattcaatattgaacgcccacacttatcggttgttggaagaagtcggtgctaccgacatgggtctgtagctcagctggttagagcaccgtcttgataaggcgggggtcgttggttcgagcccaactagacccaccaaatcttccgaacataagatgcgaggatcagtgggggattagctcagctgggagagcacctgctttgcaagcagggggtcgtcggttcgatcccgtcatcctccaccaaccaatacgctctgcggtagggcgaagaaaccaacaccaaagcggcttcgcgagaggcctctttgttgttggtccggtatagaccggatcaatcggctgttctttaaaaattcatagagtcgaatcagcgttgccggcggaaagcaggaaactgcaccgtgccgccggtgacaaaaatttgattgcgtcaaaacgaatattcaattgagcgaaagcttgttgaaattcagtaatgacgaattgttctctaggtagcaataccgaagaa

SEQ ID NO: 22 - DNA, 949 bp, Acidovorax avenae subsp. citrulli
tcgcagtctgcaactcgactgcgtgaagtcggaatcgctagtaatcgcggtcagaatgtcgcggtgaatacgttcccgggtcttgtacacaccgcccgtcacaccatgggagcgggttctgccagaagtaggtagcctaaccgtaaggagggcgcttaccacggcagggttcgtgactggggtgaagtcgtaacaaggtagccgtatcggaaggtgcggctggatcacctcctttctggaaaacagcattcaatattgaacgcccacacttatcggttgttggaagaagtcggtgctaaccgacatgggtctgtagctcagctggttagagcaccgtcttgataaggcgggggtcgttggttcgagcccaactagacccaccaaatcttccgaacataagatgcgaggatcagtgggggattagctcagctgggagagcacctgctttgcaagcagggggtcgtcggttcgatcccgtcatcctccaccaaccaatacgctctgcggtagggcgaagaaaccaacaccaaagcggcttcgcgagaggcctctttgttgttggtccggtatagaccggatcaatcggctgttctttaaaaattcatagagtcgaatcagcgttgccggcggaaagcaggaaactgcaccgtgccgccggtgacaaaaatttgattgcgtcaaaacgaatattcaattgagcgaaagcttgttgaaattcagtaatgacgaattgttctctaggtagcaataccgaagaagaattcacattacggcataacgcgcgaggtgaaagacctcgcaagtccttgaaagaaagcggagatgtctcgcaagagatgtcaaagttatagggtcaagtgactaagagcatgtggtggatgccttggcgatgataggcgacgaaagacgtgatagcctgcgataagcttcggggagctggcaaataagc

SEQ ID NO: 23 - DNA, 932 bp, Acidovorax avenae subsp. citrulli
gcttatttgccagctccccgaagcttatcgcaggctatcacgtctttcgtcgcctatcatcgccaaggcatccaccacatgctcttagtcacttgaccctataactttgacatctcttgcgagacatctccgctttctttcaaggacttgcgaggtctttcacctcgcgcgttatgccgtaatgtgaattcttcttcggtattgctacctagagaacaattcgtcattactgaatttcaacaagctttcgctcaattgaatattcgttttgacgcaatcaaatttttgtcaccggcggcacggtgcagtttcctgctttccgccggcaacgctgattcgactctatgaatttttaaagaacagccgattgatccggtctataccggaccaacaacaaagaggcctctcgcgaagccgctttggtgttggtttcttcgccctaccgcagagcgtattggttggtggaggatgacgggatcgaaccgacgaccccctgcttgcaaagcaggtgctctcccagctgagctaatcccccactgatcctcgcatcttatgttcggaagatttggtgggtctagttgggctcgaaccaacgacccccgccttatcaagacggtgctctaaccagctgagctacagacccatgtcggttagcaccgacttcttccaacaaccgataagtgtgggcgttcaatattgaatgctgttttccagaaaggaggtgatccagccgcaccttccgatacggctaccttgttacgacttcaccccagtcacgaaccctgccgtggtaagcgccctccttacggttaggctacctacttctggcagaacccgctcccatggtgtgacgggcggtgtgtacaagacccgggaacgtattcaccgcgacattctgatccgcgattactagcgattccgacttcacgcag

SEQ ID NO: 24 - DNA, 755 bp, Acidovorax avenae subsp. citrulli
cacctcctttctggaaaacagcattcaatattgaacgcccacacttatcggttgttggaagaagtcggtgctaaccgacatgggtctgtagctcagctggttagagcaccgtcttgataaggcgggggtcgttggttcgagcccaactagacccaccaaatcttccgaacataagatgcgaggatcagtgggggattagctcagctgggagagcacctgctttgcaagcagggggtcgtcggttcgatcccgtcatcctccaccaaccaatacgctctgcggtagggcgaagaaaccaacaccaaagcggcttcgcgagaggcctctttgttgttggtccggtatagaccggatcaatcgrctgttctttaaaaattcatagagtcgaatcagcgttgccggcggaaagcaggaaactgcaccgtgccgccggtgacaaaaatttgattgcgtcaaaacgaatattcaattgagcgaaagcttgttgaaattcagtaatgacgaattgttctctaggtagcaataccgaagaagaattcacattacggcataacgcgcgaggtgaaagacctcgcaagtccttgaaagaaagcggagatgtctcgcaagagatgtcaaagttatagggtcaagtgactaagagcatgtggtggatgccttggcgatgataggcgacgaaagacgtgatagcctgcgataagcttcggggagctggcaaataagctttgatccggagatttctgaatggggcaacc

SEQ ID NO: 25 - DNA, 506 bp, Ralstonia solanacearum
cacctcctttaagagcgtgcatcctagttaggcgtccacacttatcggtttgtttgatgttacagccaagggtctgtagctcaggtggttagagcaccgtcttgataaggcgggggtcgtaggttcaagtcctaccagacccaccaagttacgggcggcggagagcgatcttgccgtgaactgggggattagctcagctgggagagcacctgctttgcaagcagggggtcgtcggttcgatcccgtcatcctccaccaacaccttgtggtagccaaacgcaagcatcgagagatcggtgtttgcgtttggcattgccaagacgagtagtaactcggctgttctttaacaatatggaatgtagtaaaggtgtcgcggcgcgttgatgaggcgcgcaatttaaacgcgacactgggttgtgattgtatcaaccagtattacgagtgatcgaaagaccgcttggaatacggcacaacgcgagaactcagcctatagcgagacaaactcg

SEQ ID NO: 26 - DNA, 22 bp, artificial sequence
  primer Aac-BITS8
cggttgttggaagaagtcggtg

SEQ ID NO: 27 - DNA, 25 bp, artificial sequence
  primer Aac-BITS9
ggaagaagtcggtgctaaccgacat

SEQ ID NO: 28 - DNA, 21 bp, artificial sequence
  primer Aac-BITS10
gttggaagaagtcggtgctaa

SEQ ID NO: 29 - DNA, 20 bp, artificial sequence
  primer Aac-BITS11
aagtcggtgctaaccgacat

SEQ ID NO: 30 - DNA, 21 bp, artificial sequence
  primer Aac-BITS12
cgttttgacgcaatcaaattt

SEQ ID NO: 31 - DNA, 413 bp, Acidovorax avenae subsp. citrulli
cctctagatgcatgctcgagcggccgccagtgtgatggatatctgcagaattcgcccttaagtcgtaacaaggtacccgtatcggaaggtgcggctggatcacctcctttctggaaaacagcattcaatattgaacgcccacacttatcggttgttggaagaagtcggtgctaaccgacatgggtctgtagctcagctggttagagcaccgtcttgataaggcgggggtcgttggttcgagcccaactagacccaccaaatcttccgaacataagatgcgaggatcagtgggggattagctcagctgggagagcacctgctttgcaagcagggggtcgtcggttcgatcccgtcatcctccaccaaccaatatgtcctgcggtagggcaaagaaactaacaccaaagcggctt

SEQ ID NO: 32 - DNA, 425 bp, Acidovorax facilis
aagtcgtaacaaggtacccgtatcggaaggtgcggctggatcacctccttttgagcatgacgtcattcgtcctgtcgggcgtcctcacaaattacctgcattcagagattcataccggcacaggtcggtatgcgaagtcccttttggggccttagctcagctgggagagcacctgctttgcaagcagggggtcgtcggttcgatcccgacaggctccaccatattgagtgaaaagacttcgggtctgtagctcaggtggttagagcgcacccctgataagggtgaggtcggtagttcgagtctacccagacccaccactctgaatgtagtgcacacttaagaatttatatggatcagcgttgaggctgagacatgttcttttataacttgtgacgtagcgagcgtttgagatatctatctaaacg

SEQ ID NO: 33 - DNA, 380 bp, Psudomonas acidovorans
agtcgtaacaaggtagccgtatcggaaggtgcggctggatcacctcctttctggaaaactgctgttcaagttgaacgcccacacttatcggttgttggaacaagccatgtgccctggttgcagggtgcagtggactgggtctgtagctcagctggttagagcaccgtcttgataaggcgggggtcgttggttcgagcccaactagacccaccaagattccaatatctggttgtcgaggatcccgggggattagctcagctgggagagcacctgctttgcaagcagggggtcgtcggttcgatcccgtcatcctccaccaagatcgcgctggtggcagcggcttgaaaaagcggctggaggaagcgaaaagagcacgaaaa

SEQ ID NO: 34 - DNA, 407 bp, Acidovorax avenae subsp. citrulli
gttggaagaagtcggtgctaaccgacatgggtctgtagctcagctggttagagcaccgtcttgataaggcgggggtcgttggttcgagcccaactagacccaccaaatcttccgaacataagatgcgaggatcagtgggggattagctcagctgggagagcacctgctttgcaagcagggggtcgtcggttcgatcccgtcatcctccaccaaacgatatgctccgcggtagggcgaagaaactaacaccaaagcggcttcgcaagaggcctctttgttgttggtccggtatagaccgggtcaatcggctgttctttaaaaattcatagagtcgaatcagcgttgccggcggaaagcaggaaactgcaccgtgccgtcggcaacaaaaatttgattgcgtcaaaacg

SEQ ID NO: 35 - DNA, 796 bp, Acidovorax avenae subsp. avenae
aagtcgtaacaaggtagccgtatcggaaggtgcggctggatcacctcctttctggaaaacagcattcaatattgaacgcccacacttatcggttgttggaagaagtcggtgctaaccgacatgggtctgtagctcagctggttagagcaccgtcttgataaggcgggggtcgttggttcgagccctactagacccaccaaatcttccgaacataagatgcgaggatcagtgggggattagctcagctgggagagcacctgctttgcaagcagggggtcgtcggttcgatcccgtcatcctccaccaaccaatatgtcctgcggtagggcaaagaaactaacaccaaagcggcttcgcaagaggcctctttgttgttggtccggtatagaccgggtcaatcggctgttctttaaaaattcatagagtcgaatcagcgttgtcgacggaaagcaggaaactgcaccgtgccgtcggcaacaaaaatttgattgcgtcaaaacgaatattcaattgagcgaaagctgattgaagttcagtaatgacgaattgttctctaggtagcaataccgaagaagaattcacattacggcataacgcgcgaggtgaaagacctcgcaagtccttgaaagaaagcggagatgtctcgcaagagatgtcaaagttatagggtcaagtgactaagagcatgtggtggatgccttggcgatgataggcgacgaaagacgtgatagcctgcgataagcttcggggagctggcaaataagctttgatccggagatttctgaatggggcaacc

SEQ ID NO: 36 - DNA, 796 bp, Acidovorax avenae subsp. avenae
aagtcgtaacaaggtagccgtatcggaaggtgcggctggatcacctcctttctggaaaacagcattcaatattgaacgcccacacttatcggttgttggaagaagtcggtgctaaccgacatgggtctgtagctcagctggttagagcaccgtcttgataaggcgggggtcgttggttcgagcccaactagacccaccaaatcttccgaacataagatgcgaggatcagtgggggattagctcagctgggagagcacctgctttgcaagcagggggtcgtcggttcgatcccgtcatcctccaccaaccaatatgtcctgcggtagggcaaagaaactaacaccaaagcggcttcgcaagaggcctctttgttgttggtccggtatagaccgggtcaatcggctgttctttaaaaattcatagagtcgaatcagcgttgtcgacggaaagcaggaaactgcaccgtgccgtcggcaacaaaaatttgattgcgtcaaaacgaatattcaattgagcgaaagctgattgaagttcagtaatgacgaattgttctctaggtagcaataccgaagaagaattcacattacggcataacgcgcgaggtgaaagacctcgcaagtccttgaaagaaagcggagatgtctcgcaagagatgtcaaagttatagggtcaagtgactaagagcatgtggtggatgccttggcgatgataggcgacgaaagacgtgatagcctgcgataagcttcggggagctggcaaataagctttgatccggagatttctgaatggggcaacc

SEQ ID NO: 37 - DNA, 849 bp, Delftia acidovorans
aagtcgtaacaaggtagccgtatcggaaggtgcggctggatcacctcctttctggaaaactgctgttcaagttgaacgcccacacttatcggttgttggaacaagccatgtgccctggttgcagggtgcgtggactgggtctgtagctcagctggttagagcaccgtcttgataaggcgggggtcgttggttcgagcccaactagacccaccaagattccaatatctggttgtcgaggatcccgggggattagctcagctgggagagcacctgctttgcaagcagggggtcgtcggttcgatcccgtcatcctccaccaagatcgcgctggtggcagcggcttgaaaaagcggctggaggaagcgaaaagagcacgaaaaaagcgtgctataatatttgactcaacactaaagcagtctcgtgtgtgactgctttagtgttgatagttatccaactatcaatcggctgttctttaaaaattcatagagtcgaaatcagcgttgctgacggaaagagatttcaatctcaccgtgccgtcagcaacattttgattgcgtcaaaacgaatgaaactttgttttattcaagtaatgacgaattgttctcttgacagaaatgtcaaagaattcattcacattacggcataacgcgcgaaggtgagagacctcgcaagtccttgaaagaaaacggcgaaatctcacaagagagatcaaagttatagggtcaagtgactaagagcatgtggtggatgccttggcgatgataggcgacgaaagacgtgatagcctgcgataagcttcggggagctggcaaattagctttgatccggagatttctgaatggggcaacc

SEQ ID NO: 38 - DNA, 670 bp, Xanthomonas curcurbitae
aagtcgtaacaaggtagccgtatcggaaggtgcggctggatcacctccttttgagcatgacgtcatcgtctgcgggcgtcctcacaaattacctgcattcagagattcataccggcacaggtcggtatgcgaagtcccttttggggccttagctcagctgggagagcacctgctttgcaagcagggggtcgtcggttcgatcccgacaggctccaccatattgagtgaaaagacttcgggtctgtagctcaggtggttagagcgcacccctgataagggtgaggtcggtagttcgagtctacccagacccaccactctgaatgtagtgcacacttaagaatttatacggatcagcgttgaggctggtacgtgttcttttataacttgtgacgtagcgagcgtttgagatatctatctaaacgtgtcgttgaggctaaggcggggacttcgagtccctaagtaaattgagtcgtatgttcgcgttggtggctttgtaccccacacaacacggcatgtgaccctgaggcaacttggggttatatggtcaagcgaataagcgcacacggtggatgcctaggcggtcagaggcgatgaaggacgtggcagcctgcgaaaagtgtcggggagctggcaacaagctttgatccggcaatgtccgaatggggcaacc

SEQ ID NO: 39 - DNA, 526 bp, Erwinia tracheiphila
agtcgtaacaaggtagccgtaccggaaggtgcggctggatcacctcctttctaaggagctcaacttttggttggccacctccgtgcacgagtgttgtgcgggtggtttgctcatgggtggaatgcgatattcagtggttgcatggtggcatcgttgtggtgttgttgtgtggctgggcagagtgcactgttgggttttgaggcaatgagccgtgtggttgttgtcttggtgcctgtgcgccgtgctgggtgcctcctggttggggggtgtttggtgtggtgtggtgggttgttgtttgagaactatatagtggacgcgagcatcttggggttcctgcctgttgtgggtgggttccctggtgattgtttatgtgaatgcctttgtggtgatcatttgtgtgtaaatcgttaagggcgcacggtggatgcctgggcacaagaagccgatgaaggacgtgtgaatctgcgataagcctcggggagtcgataactgggctgtgatccgagggtgtccgaatggggcaacc

SEQ ID NO: 40 - DNA, 407 bp, Acidovorax avenae subsp. citrulli; identical to SEQ ID NO: 34

SEQ ID NO: 41 - DNA, 407 bp, Acidovorax avenae subsp. avenae
gttggaagaagtcggtgctaaccgacatgggtctgtagctcagctggttagagcaccgtcttgataaggcgggggtcgttggttcgagcccaactagacccaccaaatcttccgaacataagatgcgaggatcagtgggggattagctcagctgggagagcacctgctttgcaagcagggggtcgtcggttcgatcccgtcatcctccaccaaccaatatgtcctgcggtagggcaaagaaactaacaccaaagcggcttcgcaagaggcctctttgttgttggtccggtatagaccgggtcaatcggctgttctttaaaaattcatagagtcgaatcagcgttgtcgacggaaagcaggaaactgcaccgtgccgtcggcaacaaaaatttgattgcgtcaaaacg

SEQ ID NO: 42 - DNA, 325 bp, Acidovorax
agctggttagagcaccgtcttgataaggcgggggtcgttggttcgagcccaactagacccaccaaatcttccgaacataagatgcgaggatcagtgggggattagctcagctgggagagcacctgctttgcaagcagggggtcgtcggttcgatcccgtcatcctccaccaaccaatacgctctgcggtagggcgaagaaaccaacaccaaagcggcttcgcgagaggcctctttgttgttggtccggtatagaccggatcaatcggctgttctttaaaaattcatagagtcgaatcagcgttgccggcggaaagcaggaaactg

SEQ ID NO: 43 - protein, 16 aa, Acidovorax
DVVGAAPLTATNAAAA

(19) United States
(12) Patent Application Publication
(10) Pub. No.: US 2003/0190658 A1
(43) Pub. Date: Oct. 9, 2003

(54) Diagnostics for the detection of Acidovorax avenae subsp. citrulli, causal agent of bacterial fruit blotch in melons

(75) Inventors: James Joseph Beck, Research Triangle Park, NC (US); Charles Jason Barnett, Research Triangle Park, NC (US); Michele S. Yarnall, Research Triangle Park, NC (US); Lillian Zeitouni, Research Triangle Park, NC (US)

Correspondence Address: SYNGENTA BIOTECHNOLOGY, INC., PATENT DEPARTMENT, 3054 CORNWALLIS ROAD, P.O. BOX 12257, RESEARCH TRIANGLE PARK, NC, 27709-2257, US

(73) Assignee: Syngenta Participations AG

(21) Appl. No.: 10/397,551

(22) Filed: Mar. 25, 2003

Related U.S. Application Data
(60) Provisional application No. 60/367,628, filed on Mar. 25, 2002.

(51) Int. Cl. C12Q 1/68; C07H 21/04

(52) U.S. Cl. 435/6; 536/23.7; 536/24.3

(57) ABSTRACT

The present invention relates to diagnostic assays for the identification of Acidovorax avenae subsp. citrulli, a bacterial pathogen of melons. In particular, the present invention relates to a novel protein that is specific for A. avenae subsp. citrulli, as well as antibodies specific thereof. The invention also relates to the use of primers in polymerase chain reaction (PCR) assays for the detection of Acidovorax avenae subsp. citrulli. The use of these primers and antibodies enables the detection of specific isolates of bacterial pathogens and the monitoring of disease development in plant populations.

This application claims the benefit of U.S. Provisional Patent Application Serial No. 60/367,628 filed Mar. 25, 2002, which is incorporated herein by reference in its entirety.

### FIELD OF THE INVENTION

The present invention relates to diagnostic assays for the identification of Acidovorax avenae subsp. citrulli, a bacterial pathogen of melons. In particular, the present invention relates to a novel protein that is specific for A. avenae subsp. citrulli, as well as antibodies specific thereof. The invention also relates to the use of primers in polymerase chain reaction (PCR) assays for the detection of Acidovorax avenae subsp. citrulli. The use of these primers and antibodies enables the detection of specific isolates of bacterial pathogens and the monitoring of disease development in plant populations.

### BACKGROUND OF THE INVENTION

Diseases in plants cause considerable crop loss from year to year resulting both in economic deprivation to farmers and, in many parts of the world, to shortfalls in the nutritional provision for local populations. The widespread use of fungicides has provided considerable security against plant pathogen attack; however, despite $1 billion worth of expenditure on fungicides, worldwide crop losses amounted to approximately 10% of crop value in 1981 (James, 1981, Seed Sci. & Technol. 9: 679-685).

The severity of the destructive process of disease depends on the aggressiveness of the pathogen and the response of the host. One aim of most plant breeding programs is to increase the resistance of host plants to disease. Typically, different races of pathogens interact with different varieties of the same crop species differentially, and many sources of host resistance only protect against specific pathogen races. Furthermore, some pathogen races show early signs of disease symptoms, but cause little damage to the crop. Jones and Clifford (1983, Cereal Diseases, John Wiley) report that virulent forms of the pathogen are expected to emerge in the pathogen population in response to the introduction of resistance into host cultivars and that it is therefore necessary to monitor pathogen populations. In addition, there are several documented cases of the evolution of fungal strains that are resistant to particular fungicides. As early as 1981, Fletcher and Wolfe (1981, Proc. 1981 Brit. Crop Prot. Conf) contended that 24% of the powdery mildew populations from spring barley and 53% from winter barley showed considerable variation in response to the fungicide triadimenol and that the distribution of these populations varied between varieties, with the most susceptible variety also giving the highest incidence of less susceptible types. Similar variation in the sensitivity of fungi to fungicides has been documented for wheat mildew (also to triadimenol), Botrytis (to benomyl), Pyrenophora (to organomercury), Pseudocercosporella (to MB C-type fungicides) and Mycosphaerella fijiensis to triazoles to mention just a few (Jones and Clifford, Cereal Diseases, John Wiley, 1983).

The need for early identification of plant pathogens (A. Binder, L. Etienne, J. Beck, J. Speich & J. Youd, 1995. Practical value of crop disease diagnostic techniques. In: Hewitt et al (eds.) A vital role for fungicides in cereal production, SCI & BCPC Proceedings, UK, 231-238) has increased due to the need for judicious usage of pesticides in plant protection. Additional domains are interested in characterising the phytosanitary condition of seeds, plant material or the harvested plants. Of the numerous plant pathogens that are important in the diagnosis of plant diseases, notable ones are fungi, bacteria, viruses, viroids and phytoplasma. Which test method is used depends on the type of pathogen and the plant substrate to be examined. One method used originally to examine plant diseases was the visual evaluation of symptoms. Further examinations were normally carried out in the laboratories using microscopes or by isolating pathogens on artificial nutrients. Until a short time ago, improved examination methods were based on electron microscopy. However, electron microscopy is very time-consuming and therefore routine examinations cannot be carried out on a larger scale. A great advance was made in the development of serological examination methods based on immunological methods (F. M. Dewey & R. A. Priestley (1994): A monoclonal Antibody-based for the Detection of the Eyespot Pathogen of Cereals Pseudocercosporella herpotricoides. In Modern assays for Plant Pathogenic Fungi CAB international, 9-15) and a few disadvantages of the above-described methods could thus be eliminated.

Serological methods that are used in crop protection and are based on the enzyme-linked immunosorbent assay (ELISA) techniques are described in an overview by I. Barker (1996) (Serological methods in crop protection. In Diagnostics in Crop Protection, BCPC Proceedings, 65, 13-22).

More recently, considerable progress has been made in the development of testing methods based on DNA technology (RFLP, PCR, etc.). (J.D. Janse: (1995) New methods of diagnosis in plant pathology—perspectives and pitfalls. Bulletin OEPP/EPPO 25, 5-17).

Bacterial fruit blotch (BFB) is an economically significant disease of watermelon and muskmelon. Historically multi-million dollar lawsuits have been filed for seed sold claimed to be disease free. In 1995, BFB seriously threatened the existence of the US watermelon industry. Under the appropriate conditions the pathogen spreads rapidly through nurseries and in the field to infect melons. The pathogen is naturally borne and infects melons through seed transmission. Seed serves as the primary inoculum for BFB outbreaks (R. X. Latin and D. L. Hopkins (1995) Bacterial fruit blotch of watermelon: The hypothetical question becomes reality. Plant Dis. 79:761-765).

Currently, if a plot is thought to be infected with the disease, the organism must be cultured using traditional plating methods for positive identification. This process may take a week or longer before a conclusion can be drawn. An in-field diagnostic assay would allow early, positive identification of the disease for timely, effective disease treatment. An in-field diagnostic would facilitate monitoring seed production fields for the presence of disease. In addition, a semi-quantitative lab-based PCR assay and quantitative ELISA could be used to test seed lots for the presence of the pathogen.

### SUMMARY OF THE INVENTION

The present invention is drawn to methods of identification of different plant pathogens. The invention provides primers derived from 16S-23S rDNA spacer region sequences of Acidovorax avenae subsp. citrulli. These primers generate unique fragments in PCR reactions in which the DNA template is provided by specific bacteria and can thus be used to identify the presence or absence of a specific pathogen in host plant material before the onset of disease symptoms. The assays were used to successfully identify A. avenae subsp. citrulli isolates and differentiated them from a panel of other bacterial isolates commonly found in watermelon. The assays were also shown to cross-react with closely related species, Acidovorax avenae subsp. avenae and Acidovorax avenae subsp. cattyleae. The assays were able to detect the pathogen in infected watermelon tissue. A published PCR assay for A. avenae subsp. citrulli (Walcott et al., 2000, Plant Dis. 84:470-474) cross-reacts with A. avenae subsp. avenae, A. avenae subsp. konjaci, A. avenae subsp. cattleyae, Comonas testeronii, and an Acidovorax sp. from Calathea sp. The Syngenta developed assay has better specificity than the published assay.

Immunological methods developed include both an ELISA based format and lateral flow strip format (immunostrip) for the detection of Acidovorax avenae subsp. citrulli. ELISA based methods provided less than 0.2% cross-reactivity among the bacterial species investigated with the exception of Acidovorax avenae subsp. avenae. Immunostrips were negative for cross-reactivity against these same strains, though slight positives were observed with Acidovorax avenae subsp. avenae and Acidovorax avenae subsp. konjaci. Neither A. avenae subsp. avenae nor A. avenae subsp. konjaci is known to be pathogenic to melons. Immunostrips had good sensitivity to field samples of watermelon suspected of disease and band intensity of the immunostrips correlated roughly with ELISA results on the same materials.

Despite the lack of absolute specificity of the immunostrip and PCR assays for A. avenae subsp. citrulli, the risk of false positive detection is minimal due to the absence of other A. avenae subspecies in melon seeds. The A. avenae subspecies konjaci, cattleyae and avenae are not known to cause diseases on cucurbits and were not previously found associated with cucurbit hosts.

Thus, the present invention provides for a nucleic acid molecule encoding a 16S-23S spacer DNA sequences for the bacterial species Acidovorax avenae subsp. citrulli, Acidovorax avenae subsp. avenae, Xanthomonas curcurbitae, and Erwinia tracheiphila. In a preferred embodiment, the nucleic acid molecules wherein the 16S-23S spacer DNA sequence is SEQ ID NOS: 5, 12-24, 31, 34-36, or 38-42.

The invention also provides for a nucleic acid molecule having sequence identity with at least 10 contiguous nucleotides of the 16S-23S rDNA spacer sequence from Acidovorax avenae subsp. citrulli In a more particular embodiment, the nucleic acid molecule wherein the 16S-23S rDNA spacer sequence has the sequence of SEQ ID NOS: 5, 12-24, 34, or 40. Prefereably, the nucleic acid molecule comprises a nucleotide sequence of SEQ ID NOs: 2-4,6-11 or 26-30.

The invention also provides for a pair of oligonucleotide primers wherein at least one primer consists of the nucleotide sequence of SEQ ID NOS: 2-4, 6-11 or 26-30. In a more particular embodiment, the pair of oligonucleotide primers comprises Aac-BITS 10 (SEQ ID NO: 28) and Aac-BITS12 (SEQ ID NO: 30).

The invention further provides for a method for the detection of a bacterial pathogen, comprising the steps of:

(a) isolating DNA from a plant tissue infected with a pathogen;

(b) subjecting said DNA to polymerase chain reaction amplification using at least one primer having sequence identity with at least 10 contiguous nucleotides of a 16S-23S rDNA spacer region sequence of a Acidovorax spp.; and

(c) detecting said bacterial pathogen by visualizing the product or products of said polymerase chain reaction amplification.

More particularly, the method is for detecting the bacterial pathogen is Acidovorax avenae subsp. citrulli. In a preferred embodiment, the method has the 16S-23S spacer sequences have the nucleotide sequence of SEQ ID NO: 24. In another preferred embodiment, at least one primer having the nucleotide sequence of SEQ ID NOS: 2-14.

The invention also provides a method for the detection of a bacterial pathogen, comprising the steps of:

(a) isolating DNA from a plant tissue infected with a pathogen;

(b) subjecting said DNA to polymerase chain reaction amplification using at least one primer having sequence identity with at least 10 contiguous nucleotides of a 16S-23S rDNA spacer sequence of Acidovorax avenae subsp. citrulli; and

(c) detecting said bacterial pathogen by visualizing the product or products of said polymerase chain reaction amplification.

In a preferred embodiment, the bacterial pathogen is Acidovorax avenae subsp. citrulli. In another preferred embodiment the uses at least one primer having the nucleotide sequence of SEQ ID NOS: 2-4, 6-11, or 26-30. In a more preferred embodiment, the method uses a pair of oligonucleotide primers consists of SEQ ID NO: 28 and SEQ ID NO: 30.

The invention also provides for a diagnostic kit used in detecting a bacterial pathogen comprising at least one primer having at least 10 contiguous nucleotides of a 16S and 16S-23S rDNA spacer sequence of Acidovorax avenae subsp. citrulli. In a preferred embodiment, at least one primer of SEQ ID NOs: 2-4,6-11 and 26-30 for 16S and 16S-23S rDNA spacer derived primers. In more preferred embodiment, the pair of primers are SEQ ID NO: 28 and SEQ ID NO: 30.

The invention also provides for a polypeptide comprising the amino acid sequence of DVVGAAPLTATNAAAA (SEQ ID NO: 43). The invention also provides for an antibody that reacts with a polypeptide having the N-terminal amino acid sequence of SEQ ID NO: 43.

The invention provides for an immunoassay for the detection of Acidovorax avenae subsp. citrulli that uses the antibody that reacts with a polypeptide having the N-terminal amino acid sequence of SEQ ID NO: 43. In preferred embodiments, the immunoassay is an ELISA or lateral flow strip format. In a more preferred embodiment, the immunoassay is used to detect the presence of Acidovorax avenae subsp. citrulli in cucurbit hosts.

the invention also provides for a kit for the detection by the immunoassay comprising a carrier being compartmented to receive in close confinement therein:

(a) a means of extraction of a test substance in the presence of a primary antibody capable of binding to the test substance wherein said primary antibody is conjugated to a means of detection;

(b) solid phase format having a significant measurement in three dimensions to form a substantial volume with a plurality of interstitial spaces capable of capturing a complex formed by the primary antibody and the test substance;

(c) a vessel containing a buffer;

(d) reagents reactive with the means of detection to produce a detectable reaction product; and

(e) a means of dispensing said reagents.

BRIEF DESCRIPTION OF THE SEQUENCES IN THE SEQUENCE LISTING

| SEQ ID NO:1 | Acidovorax avenae subsp. citrulli 16S ribosomal RNA gene, partial | | |
|---|---|---|---|
| | sequence GenBank Accession Number AF137506. | | |
| SEQ ID NO:2 | Oligonucleotide Primer | BITS-1 | From Söller et. al. Int. J. Syst. Evol. |
| | | | Micro. 2000, 50, 909-915. |
| SEQ ID NO:3 | Oligonucleotide Primer | Aac-BITS1 | BITS-1 sequence modified to |
| | | | amplify from Acidovorax spp. |
| SEQ ID NO:4 | Oligonucleotide Primer | BITS-2 | From Söller et. al. Int. J. Syst. Evol. |
| | | | Micro. 2000, 50, 909-915. |
| SEQ ID NO:5 | Acidovorax avenae subsp. citrulli, isolate 29625, partial sequence of PCR | | |
| | product amplified using primers BITS-1 and BITS-2 in the 16S-23S | | |
| | ribosomal DNA spacer region; Syngenta Identifier 21 Aug00F. | | |
| SEQ ID NO:6 | Oligonucleotide Primer | Aac-BITS3 | |
| SEQ ID NO:7 | Oligonucleotide Primer | Aac-BITS4 | |
| SEQ ID NO:8 | Oligonucleotide Primer | Aac-BITS6 | |
| SEQ ID NO:9 | Oligonucleotide Primer | Aac-BITS5 | |
| SEQ ID NO:10 | Oligonucleotide Primer | Aac-BITS7 | |
| SEQ ID NO:11 | Oligonucleotide Primer | 1100F | Modified from primer 1100r in Lane, |
| | | | D. J. “16S/23S rRNA sequencing” |
| | | | Nucleic acid techniques in bacterial |
| | | | systematics. Stackebrandt and |
| | | | Goodfellow eds. 1991. John Wiley |
| | | | and Sons, England, p 133. |

| SEQ ID NO:12 | Acidovorax avenae subsp. citrulli, isolate 29625, sequence of the 16S- | |
|---|---|---|
| | 23S ribosomal DNA spacer region between primers 1100F and Aac- | |
| | BITS6; Syngenta Identifier Sequences pCRAacD2-1. | |
| SEQ ID NO:13 | Acidovorax avenae subsp. citrulli, isolate 29625, sequence of the 16S- | |
| | 23S ribosomal DNA spacer region between primers 1100F and Aac- | |
| | BITS6; Syngenta Identifier Sequences | |
| | pCRAacD4-1. | |
| SEQ ID NO:14 | Acidovorax avenae subsp. citrulli, isolate 29625, sequence of the 16S- | |
| | 23S ribosomal DNA spacer region between primers AacBITS-7 and | |
| | BITS2; Syngenta Identifier pCRAAC29625-C2-17. | |
| SEQ ID NO:15 | Acidovorax avenae subsp. citrulli, isolate 29625, sequence of the 16S- | |
| | 23S ribosomal DNA spacer region between primers AacBITS-7 and | |
| | BITS2; Syngenta Identifier pCRAAC29625-C4-22. | |
| SEQ ID NO:16 | Acidovorax avenae subsp. citrulli, isolate 29625, sequence of the 16S- | |
| | 23S ribosomal DNA spacer region between primers AacBITS-7 and | |
| | BITS2, consensus sequence obtained by comparing sequences | |
| | identified as SEQ ID NOs: 14 and 15; Syngenta Identifier 29625BIT5. | |
| SEQ ID NO:17 | Acidovorax avenae subsp. citrulli, isolate zucchini #6, sequence of the | |
| | 16S-23S ribosomal DNA spacer region between primers AacBITS-7 | |
| | and BITS2; Syngenta Identifier pCRAACz-13. | |
| SEQ ID NO:18 | Acidovorax avenae subsp. citrulli, isolate yellow squash, sequence of | |
| | the 16S-23S ribosomal DNA spacer region between primers AacBITS- | |
| | 7 and BITS2; Syngenta Identifier pCRAACysq-5. | |
| SEQ ID NO:19 | Acidovorax avenae subsp. citrulli, isolate lA cantaloupe, sequence of | |
| | the 16S-23S ribosomal DNA spacer region between primers AacBITS- | |
| | 7 and BITS2; Syngenta Identifier pCRAAC-IA-CANT-1. | |
| SEQ ID NO:20 | Acidovorax avenae subsp. citrulli, isolate 94-21, sequence of the 16S- | |
| | 23S ribosomal DNA spacer region between primers AacBITS-7 and | |
| | BITS 2; Syngenta Identifier pCRAAC94-21-9. | |
| SEQ ID NO:21 | Acidovorax avenae subsp. citrulli, isolate 92-17, partial sequence of | |
| | the PCR product amplified using primers Aac-BITS7 and BITS2 in the | |
| | 16S-23S ribosomal DNA spacer region; Syngenta Identifier | |
| | 23Oct00A3. | |
| SEQ ID NO:22 | Acidovorax avenae subsp. citrulli, isolate Au-9, partial sequence of the | |
| | PCR product amplified using primers Aac-BITS7 and BITS2 in the | |
| | 16S-23S ribosomal DNA spacer region; Syngenta Identifier | |
| | 23Oct00A4. | |
| SEQ ID NO:23 | Acidovorax avenae subsp. citrulli, isolate 98-16, partial sequence of | |
| | the PCR product amplified using primers Aac-BITS7 and BITS2 in the | |
| | 16S-23S ribosomal DNA spacer region; Syngenta Identifier | |
| | 23Oct00A5. | |
| SEQ ID NO:24 | Syngenta sequence 29625BIT5 (SEQ ID NO:16) truncated by | |
| | removing 16S rDNA sequence to leave only 16S-23S region spacer | |
| | sequence for Acidovorax avenae subsp. citrulli, isolate 29625; | |
| | Syngenta Identifier 29625BIT5 minus 16S. | |
| SEQ ID NO:25 | Ralstonia solanacearum 16S ribosomal RNA gene, partial sequence | |
| | GenBank Accession Number AJ277856. | |
| SEQ ID NO:26 | Oligonucleotide Primer | Aac-BITS8 |
| SEQ ID NO:27 | Oligonucleotide Primer | Aac-BITS9 |
| SEQ ID NO:28 | Oligonucleotide Primer | Aac-BITS10 |
| SEQ ID NO:29 | Oligonucleotide Primer | Aac-BITS11 |
| SEQ ID NO:30 | Oligonucleotide Primer | Aac-BITS12 |

| | |
|---|---|
| SEQ ID NO:31 | Acidovorax avenae subsp. avenae, isolate 78-5, partial sequence of the |
| | PCR product amplified using primers Aac-BITS7 and BITS2 in the |
| | 16S-23S ribosomal DNA spacer region; Syngenta Identifier |
| | 15May01.5.AAA. |
| SEQ ID NO:32 | Acidovorax facilis, isolate 94-1, partial sequence of the PCR product |
| | amplified using primers Aac-BITS7 and BITS2 in the 16S-23S |
| | ribosomal DNA spacer region; Syngenta Identifier 15May01.5.AAA. |
| SEQ ID NO:33 | Pseudomonas acidovorans, isolate ATCC 15669, sequence of the PCR |
| | product amplified using primers Aac-BITS1 and BITS2 in the 16S-23S |
| | ribosomal DNA spacer region; Syngenta Identifier |
| | 15May01.9.Pacidovorans. |
| SEQ ID NO:34 | Acidovorax avenae subsp. citrulli, isolate 33619, sequence of the PCR |
| | product amplified using primers BITS1 and BITS2 in the 16S-23S |
| | ribosomal DNA spacer region; Syngenta Identifier BIT533619I-19. |
| SEQ ID NO:35 | Acidovorax avenae subsp. avenae, isolate 19307, sequence of the PCR |
| | product amplified using primers BITS1 and BITS2 in the 16S-23S |
| | ribosomal DNA spacer region; Syngenta Identifier BIT519307E-12. |
| SEQ ID NO:36 | Acidovorax avenae subsp. avenae, isolate 78-5, sequence of the PCR |
| | product amplified using primers BITS1 and BITS2 in the 16S-23S |
| | ribosomal DNA spacer region; Syngenta Identifier BITSAaa78-5F-14. |
| SEQ ID NO:37 | Delftia acidovorans, isolate 15668, sequence of the PCR product |
| | amplified using primers BITS1 and BITS2 in the 16S-23S ribosomal |
| | DNA spacer region; Syngenta Identifier BITSDacidD-8. |
| SEQ ID NO:38 | Xanthomonas curcurbitae, isolate 23378, sequence of the PCR product |
| | amplified using primers BITS1 and BITS2 in the 16S-23S ribosomal |
| | DNA spacer region; Syngenta Identifier BITSXcampB-4. |
| SEQ ID NO:39 | Erwinia tracheiphila, isolate 27003, sequence of the PCR product |
| | amplified using primers BITS1 and BITS2 in the 16S-23S ribosomal |
| | DNA spacer region; Syngenta Identifier BITSEtrachA-3. |
| SEQ ID NO:40 | Acidovorax avenae subsp. citrulli, isolate 33619, sequence of the PCR |
| | product amplified using primers Aac-BITS10 and Aac-BITS12 in the |
| | 16S-23S ribosomal DNA spacer region; Syngenta Identifier |
| | Aac33619I-19. |
| SEQ ID NO:41 | Acidovorax avenae subsp. avenae, isolate 78-5, sequence of the PCR |
| | product amplified using primers Aac-BITS10 and Aac-BITS12 in the |
| | 16S-23S ribosomal DNA spacer region; Syngenta Identifier |
| | AacAaa78-5J. |
| SEQ ID NO:42 | Sequence of the PCR product amplified using primers Aac-BITS10 |
| | and Aac-BITS12 from a DNA extraction made of Watermelon Sample |
| | N; Syngenta Identifier Watermelon N Aac 10 12 product. |
| SEQ ID NO:43 | N-terminal sequence from 160 kDa protein unique to Acidovorax spp. |

### DETAILED DESCRIPTION OF THE INVENTION

The present invention provides unique DNA sequences that are useful in identifying different pathotypes of plant pathogenic fungi. Particularly, the DNA sequences can be used as primers in PCR-based analysis for the identification of bacterial pathotypes. The DNA sequences of the invention include primers derived from partial sequences of the 16S rDNA and 16S-23S rDNA spacer regions of particular bacterial pathogens that are capable of identifying the particular pathogen.

Biomedical researchers have used PCR-based techniques for some time and with moderate success to detect pathogens in infected animal tissues. Only recently, however, has this technique been applied to detect plant pathogens. The presence of Gaumannomyces graminis in infected wheat has been detected using PCR of sequences specific to the pathogen mitochondrial genome (Schlesser et al, 1991, Applied and Environ. Microbiol. 57: 553-556), and random amplified polymorphic DNA (i.e. RAPD) markers were able to distinguish numerous races of Gremmeniella abietina, the causal agent of scleroderris canker in conifers. U.S. Pat. No. 5,585,238 (herein incorporated by reference in its entirety) describes primers derived from the ITS sequences of the ribosomal RNA gene region of strains of Septoria, Pseudocercosporella, and Mycosphaerella and their use in the identification of these fungal isolates using PCR-based techniques. In addition, U.S. Pat. No. 5,955,274 (herein incorporated by reference in its entirety) describes primers derived from the ITS sequences of the ribosomal RNA gene region of strains of Fusarium and their use in the identification of these fungal isolates using PCR-based techniques. Furthermore, U.S. Pat. No. 5,800,997 (herein incorporated by reference in its entirety) describes primers derived from the ITS sequences of the ribosomal RNA gene region of strains of Cercospora, Helminthosporium, Kabatiella, and Puccinia and their use in the identification of these fungal isolates using PCR-based techniques.

Ribosomal genes are suitable for use as molecular probe targets because of their high copy number. Despite the high conservation between mature rRNA sequences (for example the 16S rRNA gene), the spacer sequences between them are usually poorly conserved and are thus suitable as target sequences for the detection of recent evolutionary divergence. Bacterial rRNA genes are organized in units, each of which encodes three mature subunits of 16S (small subunit), 5S, and 28S (large subunit) as well as either one or two tRNA genes. The 16S and 23S rRNA subunits are separated by a 16S-23S spacer region. The use of the divergences found in bacterial 16S-23S spacer regions for purposes of typing and identification is discussed thoroughly by Gürtler and Stanisich (Microbiology. 1996, 142, 3-16.). The divergences found in the 16S-23S spacer region sequences are particularly suitable for the detection of specific species or subspecies of different bacterial pathogens.

The DNA sequences of the invention are from partial sequences of 16S rRNA gene the 16S-23S spacer regions of different bacteria. The DNA sequences of these regions from different species or subspecies within a pathogen species or genus vary among the different members of the species or genus. Once the sequences of either of these regions have been determined for a given pathogen, they can be aligned with other sequences from the same region for other pathogens. In this manner, primers can be derived from the 16S rRNA gene and/or the 16S-23S spacer regions that are specific for a given pathogen at some level of taxonomy. That is, primers can be designed based on regions within either the 16S rRNA gene or the 16S-23S spacer region sequences that contain the greatest differences in sequence among the bacterial pathotypes when similar regions are compared. These sequences and primers based on these sequences can be used to identify specific pathogens.

In a preferred embodiment, the invention provides novel 16S-23S spacer DNA sequences for the bacterial species Acidovorax avenae subsp. citrulli, Acidovorax avenae subsp. avenae, Xanthomonas curcurbitae, and Erwinia tracheiphila.

The present invention provides oligonucleotide primers for use in amplification-based detection of a bacterial 16S-23S rDNA spacer sequence, wherein said primer has sequence identity with at least 10 contiguous nucleotides of the 16S-23S rDNA spacer sequence from Acidovorax avenae subsp. citrulli.

In preferred embodiments, oligonucleotide primers derived from the 16S rDNA or 16S-23S rDNA spacer sequences comprise or consist of a nucleotide sequence of SEQ ID NOs: 2-4, 6-11, 8-12 and 26-30. The primers are useful in the PCR-based identification of Acidovorax avenae subsp. citrulli.

In a preferred embodiment, the invention provides a pair of oligonucleotide primers wherein at least one primer consists of the nucleotide sequence of SEQ ID NOS: 2-4, 6-11, 8-12 and 26-30. A preferred pair of primers is Aac-BITS10(SEQ ID NO: 28) and Aac-BITS12 (SEQ ID NO: 30).

The present invention is also drawn to immunodiagnostic tools for the identification of plant pathogens. The invention provides a protein unique to the plant pathogen A. avenae subsp. citrulli. In a preferred embodiment, the invention provides the protein unique to A. avenae subsp. citrulli is an approximately 160 kDa protein with an N-terminal amino acid sequence of DVVGAAPLTATNAAAA (SEQ ID NO: 43).

The protein of this invention is useful in the method of this invention as it is ued to create polyclonal and monoclonal antibodies for use in immunodiagnostic assays. These antibodies are specific to certain bacteria and can be used to identify the presence of these pathogens in host plant tissues. Therefore, the invention provides an antibody that reacts with a protein unique to the plant pathogen, A. avenae subsp. citrulli. In a preferred embodiment, the invention provides an antibody that reacts with a 160 kDa protein unique to A. avenae subsp. citrulli with an N-terminal amino acid sequence of DVVGAAPLTATNAAAA (SEQ ID NO: 43).

The present invention provides an immunostrip and ELISA immunoassays that use an antibody that reacts only with a protein unique to the the plant pathogen A. avenae subsp. citrulli. In a preferred embodiment, the invention provides an immunostrip and ELISA immunoassays that use an antibody that reacts with a 160 kDa protein unique to the plant pathogen A. avenae subsp. citrulli with an N-terminal amino acid sequence of DVVGAAPLTATNAAAA (SEQ ID NO: 43).

Methods for the use of the primer sequences of the invention in PCR analysis are well known in the art. For example, see U.S. Pat. Nos. 4,683,195 and 4,683,202, as well as Schlesser et al. (1991) Applied and Environ. Microbiol. 57:553-556. See also, Nazar et al. (1991, Physiol. and Molec. Plant Pathol. 39: 1-11), which used PCR amplification to exploit differences in the ITS regions of Verticillium albo-atrum and Verticillium dahliae and therefore distinguish between the two species; and Johanson and Jeger (1993, Mycol. Res. 97: 670-674), who used similar techniques to distinguish the banana pathogens Mycosphaerella fijiensis and Mycosphaerella musicola.

The target DNA sequences of the invention can be cloned from bacterial pathogens by methods known in the art. In general, the methods for the isolation of DNA from bacterial isolates are known (J. Sambrook, E. F. Fritsch and T. Maniatis, Molecular Cloning: A Laboratory manual, Cold Spring Harbor laboratory, Cold Spring Harbor, N.Y. (1989).

The 16S-23S rDNA spacer sequences are compared within each pathogen group to locate divergences that might be useful to test in PCR to distinguish the different species and/or subspecies. From the identification of divergences, numerous primers are synthesized and tested in PCR-amplification. Templates used for PCR-amplification testing are firstly purified pathogen DNA, and subsequently DNA isolated from infected host plant tissue. Thus, it is possible to identify pairs of primers that are diagnostic, i.e. that identified one particular pathogen species or strain but not another species or strain of the same pathogen. Primers are also designed to regions highly conserved among the species to develop genus-specific primers as well as primers that will identify any of several bacterial pathogens that cause a particular disease. For example, primers are developed to differentiate subspecies of Acidovorax avenae.

Preferred primer combinations are able to distinguish between the different species or strains in infected host tissue, i.e. host tissue that has previously been infected with a specific pathogen species or strain. This invention provides numerous primer combinations that distinguish Acidovorax avenae subsp. citrulli. The primers of the invention are designed based on sequence differences among either the 16S or 16S-23S rDNA spacer regions. A minimum of one base pair difference between sequences can permit design of a discriminatory primer. Primers designed to a specific bacterial DNA sequence can be used in combination with a primer made to a conserved sequence region flanking the region containing divergences to amplify species-specific PCR fragments. In general, primers should have a theoretical melting temperature between about 60 to about 70 degree ° C. to achieve good sensitivity and should be void of significant secondary structure and 3′ overlaps between primer combinations. In preferred embodiments, primers are anywhere from approximately 5-30 nucleotide bases long.

In one embodiment, the present invention provides a method for the detection of a bacterial pathogen, comprising the steps of:

(a) isolating DNA from a plant tissue infected with a pathogen;

(b) subjecting said DNA to polymerase chain reaction amplification using at least one primer having sequence identity with at least 10 contiguous nucleotides of a 16S-23S rDNA spacer region sequence of a Acidovorax spp.; and

(d) detecting said bacterial pathogen by visualizing the product or products of said polymerase chain reaction amplification.

In preferred embodiments, the method detects infections with a pathogen, wherein said bacterial pathogen is Acidovorax avenae subsp. citrulli. In another preferred embodiment, the 16S-23S spacer sequences have the nucleotide sequence of SEQ ID NO: 24.

In another preferred embodiment, the method uses at least one primer having the nucleotide sequence of SEQ ID NOS: 2-14. In another embodiment, the present invention provides for a method for the detection of a bacterial pathogen, comprising the steps of:

(a) isolating DNA from a plant tissue infected with a pathogen;

(b) subjecting said DNA to polymerase chain reaction amplification using at least one primer having sequence identity with at least 10 contiguous nucleotides of a 16S-23S rDNA spacer sequence of Acidovorax avenae subsp. citrulli; and

(c) detecting said bacterial pathogen by visualizing the product or products of said polymerase chain reaction amplification.

In preferred embodiments, the method detects the bacterial pathogen Acidovorax avenae subsp. citrulli.

In another preferred embodiment, the method uses at least one primer having the nucleotide sequence of SEQ ID NOS: 2-4,6-11, and 26-30.

In more preferred embodiments, the methods uses a pairs of oligonucleotide primers wherein said pair consists of SEQ ID NO: 28 and SEQ ID NO: 30.

The present invention lends itself readily to the preparation of “kits” containing the elements necessary to carry out the process. Such a kit may comprise a carrier being compartmentalized to receive in close confinement therein one or more container, such as tubes or vials. One of the containers may contain unlabeled or detectably labeled DNA primers. The labeled DNA primers may be present in lyophilized form or in an appropriate buffer as necessary. One or more containers may contain one or more enzymes or reagents to be utilized in PCR reactions. These enzymes may be present by themselves or in admixtures, in lyophilized form or in appropriate buffers.

In one embodiment, the diagnostic kit used in detecting a bacterial pathogen, comprises at least one primer of SEQ ID NOs: 2-4,6-11.and 8-12 for 16S and 16S-23S rDNA spacer derived primers.

In more preferred embodiments, the diagnostic kit used in detecting a bacterial pathogen, comprises the pair of primers described above. More preferably, the pairs of primers are SEQ ID NO: 28 and SEQ ID NO: 30.

Finally, the kit may contain all of the additional elements necessary to carry out the technique of the invention, such as buffers, extraction reagents, enzymes, pipettes, plates, nucleic acids, nucleoside triphosphates, filter paper, gel materials, transfer materials, autoradiography supplies, and the like.

The examples below show typical experimental protocols that can be used in the selection of suitable primer sequences, the testing of primers for selective and diagnostic efficacy, and the use of such primers for disease and bacterial isolate detection. Such examples are provided by way of illustration and not by way of limitation.

Numerous references cited above are all incorporated herein in their entireties.

### EXAMPLES

Standard recombinant DNA and molecular cloning techniques used here are well known in the art and are described by J. Sambrook, E. F. Fritsch and T. Maniatis, Molecular Cloning: A Laboratory manual, Cold Spring Harbor laboratory, Cold Spring Harbor, N.Y. (1989) and by T. J. Silhavy, M. L. Berman, and L. W. Enquist, Experiments with Gene Fusions, Cold Spring Harbor Laboratory, Cold Spring Harbor, N.Y. (1984) and by Ausubel, F. M. et al., Current Protocols in Molecular Biology, pub. by Greene Publishing Assoc. and 20 Wiley-Interscience (1987).

### Example 1

### Bacterial Isolates and Genomic Bacterial DNA Extraction

See Table 1 for listing of the bacterial isolates used and their sources. Isolates used to validate the assays in the following examples were obtained from a number of academic institutions and collections (Table 1).

TABLE 1
Source of Test Isolates

| | Isolate | |
|---|---|---|
| Species | Designation | Source |
| Acidovorax avenae subsp. citrulli | 94-21 | Walcott¹ |
| Acidovorax avenae subsp. citrulli | Zucchini | Walcott¹ |
| Acidovorax avenae subsp. citrulli | Yellow squash | Walcott¹ |
| Acidovorax avenae subsp. citrulli | IA Cantaloupe | Walcott¹ |
| Acidovorax avenae subsp. citrulli | 92-17 | Walcott¹ |
| Acidovorax avenae subsp. citrulli | Au-9 | Walcott¹ |
| Acidovorax avenae subsp. citrulli | 29625 | ATCC² |
| Acidovorax avenae subsp. avenae | 19307 | ATCC² |
| Acidovorax avenae subsp. avenae | 78-5 | Walcott¹ |
| Acidovorax avenae subsp. cattyleae | 33619 | ATCC² |
| Acidovorax avenae subsp. cattyleae | 98-1 | Walcott¹ |
| Acidovorax avenae subsp. avenae | 78-5 | Walcott¹ |
| Acidovorax avenae subsp. konjaci | 33996 | ATCC² |
| Delftia acidovorans | 15668 | ATCC² |
| Erwinia tracheiphila | 27003 | ATCC² |
| Xanthomonas curcurbitae | 23378 | ATCC² |
| Agrobacterium radiobacter | K84 | Gonzalez³ |
| Agrobacterium radiobacter | 79-1 | Walcott¹ |
| Raistonia solanacearum | K60 | Gonzalez³ |
| Pseudomonas phaseolicola | 00-1 | Gonzalez³ |
| Xanthomonas campestris pv. campestris | 00-1 | Walcott¹ |
| Pantoea agglomerans | 99-3 | Walcott¹ |
| Burkholderia cepacia | 92-1 | Walcott¹ |
| Pseudomonas viridiflava | 00-1 | Walcott¹ |
| Pseudomonas marginata | 83-1 | Walcott¹ |
| Acidovorax facilis | 94-1 | Walcott¹ |
| Xanthomonas campestris pv. vesicatoria | 00-1 | Walcott¹ |
| Pseudomonas aeruginosa | 84-1 | Walcott¹ |
| Enterobacter cloacae | | Kloepper^{4*} |
| Burkholderia gladioli pv gladioli | | Kloepper^{4*} |

¹Dr. Ronald Walcott; Dept. of Plant Pathology; University of Georgia; Athens, GA, USA
²American Type Culture Collection; Rockville, MD, USA
³Dr. Carlos Gonzalez, Department of Plant Pathology and Microbiology; Texas A&M University; College Station, TX, USA
⁴Dr. Joe Kloepper, Dept. of Plant Pathology, Auburn University; Auburn SC, USA
^{*}isolate from nature, identified by Fatty Acid Methyl Ester analysis

### Example 2

### Bacterial Genomic DNA Extractions

Unless cultures are obtained from the American Type Culture Collection (ATCC, as indicated in Table 1), bacterial cultures are received in a 0.03% sodium azide solution and are non-viable. DNA is extracted from the viable stocks by first growing fresh cultures on nutrient enriched agar and harvesting a loopful of cells. A FastDNA kit (QbioGene, Carlsbad, Calif., U.S.A.) is used for the DNA extraction according to manufacturer's instructions for obtaining DNA from bacteria. Cells from the non-viable stocks are pelleted by taking ˜500 μl of cell suspension and centrifuging at 6,000 r.p.m. for 5 minutes. The sodium azide supernatant solution is removed and cells are resuspended in sterile saline EDTA (150 mM NaCl, 100 mM EDTA, pH 8). The cells are then re-pelleted at 3,000 rpm for 5 minutes and the wash removed. The pellet is resuspended in 1 ml proteinase K buffer (0.01 M Tris/HCl, 0.005 M EDTA, 0.5% SDS, pH 7.8) and 15 μl of proteinase K (20 mg/ml) and 5.5 μl of 22.7% w/v SDS are added. The bacteria are incubated in this mixture for one hour. Then 55 μl of 22.7% SDS and 40 μl of 5 M NaCl are added. This mixture is extracted by an equal volume 25:24:1 phenol:chloroform:isoamyl alcohol extraction. The extraction is vortexed and spun at 10,000 rpm for 10 minutes. The aqueous layer is transferred to a fresh tube. DNA is precipitated by the addition of 0.1 volume of 5 M sodium acetate pH 5.2 and by filling the rest of the 1.5 ml tube (with space for closing cap) with isopropanol. This precipitation is mixed by inverting several times and placed in a −20° C. freezer for one hour. The precipitated DNA is pelleted by centrifuging at 12,000 rpm for 10 minutes. The supernatant is discarded and the pellet washed with 0.5 mL of 70% ethanol. The pellet is once more spun down, the wash discarded and the pellet is allowed to dry by placing the tube in a dessicator. The pellet is resuspended in 200 uL of TE buffer with RNase A (10 μg/ml) and the DNA concentration read on a UV-spectrophotometer. 10 ng/μL dilutions of DNA are prepared for use in all PCR reactions.

### Example 3

### Bacterial Protein Extractions

Bacterial extracts made from the same material as above are prepared using the Pierce (Rockford, Ill., USA) B-PER® bacterial protein extraction reagent and following the manufacturer's recommended protocol.

### Example 4

### Source of Watermelon Tissues

Shipments of watermelon field samples are received from fields in Arkansas, USA in individually wrapped plastic bags and kept refrigerated prior to use in testing (Table 2).

TABLE 2
Field-grown watermelon tissues

| Sample Designation | Variety | Visual Assessment |
|---|---|---|
| A | Stars & Stripes | Healthy leaves? |
| B | Stars & Stripes | Diseased leaves? |
| C | Stars & Stripes | Diseased leaves? |
| D | Stars & Stripes | Healthy-looking rind |
| E | Stars & Stripes | Diseased rind? |
| F | Carousel | Healthy Leaves? |
| G | Carousel | Healthy Leaves? |
| H | Carousel | Diseased Leaves |
| I | Carousel | Diseased Leaves |
| J | Carousel | Diseased Leaves |
| K | Carousel | Diseased Leaves |
| L | Carousel | Diseased Leaves |
| Ml | Carousel | Healthy rinds? |
| M2 | Carousel | Healthy rinds? |
| N | Carousel | Diseased rinds |
| O | Carousel | Diseased rinds |
| P | Carousel | Diseased rinds |
| Q | Carousel | Diseased rinds |
| R | Carousel | Diseased rinds |
| S | Fandango | Healthy Leaves? |
| T | Fandango | Diseased Leaves |
| U | Fandango | Diseased rinds |
| V | Fandango | Diseased rinds |
| W | Sugartime | Healthy Leaves? |
| X | Sugartime | Diseased Leaves |
| Y | Sugartime | Diseased rinds |

Seedlings experimentally inoculated with A. avenae subsp. citrulli are also received (Table 3). Several hundred seedlings are shipped in large zip-lock bags separated according to amount of positive seedlings in each bag. Seedling samples come from a grow-out of several commercial seed lots that were either positive or negative for bacterial fruit blotch. Seedlings from two trays planted with 500 seeds grown for three weeks are harvested for each population. There are approximately 700-800 seedlings per population. The description of these populations is documented in Table 3.

TABLE 3
Descriptions of experimentally inoculated seedling populations

| Seedling Batch | Description |
|---|---|
| A | Negative for Aac |
| B | Mostly negative, one symptomatic seedling |
| C | Mostly negative, ten symptomatic seedlings |
| D | Thirty percent of seedlings symptomatic |

Syngenta Seeds also arranged for the shipment of seed batches infected with A. avenae subsp. citrulli. Table 4 summarizes the seed batches received. Seeds are maintained at room temperature in their original shipping container, either a seed envelope or cloth bag.

TABLE 4
Seed samples

| Seed Identification | Description from other labs |
|---|---|
| Control | Healthy |
| T700 + PV0110 | Blend of positive seed batch |
| PV0110 | Gave consistent grow outs |
| PV0113 | Gave marginal grow outs |
| Watermelon 6E51 | Gave consistent grow outs |
| Cantaloupe SA123 | Gave consistent grow outs |

Note:
“grow outs” indicates A. avenae subsp. citrulli was cultured from the seeds.
Example 5: Watermelon tissue protein extraction

Watermelon leaves, approximately 1″ square, are extracted in 500 μl of extraction buffer in a 1.5 ml conical tube using a disposable pestle. The tissue is extracted for about 30 see or until the tissue appears macerated. For fruit testing, pieces of tissue approximately ¼″×½″×¼″ are cut directly from under the rind and extracted in 500 μl of extraction buffer as described above.

Testing of seedling samples requires larger amounts of material. Fifty seedlings are placed in an extraction bag with 30 ml of extraction buffer and extracted until the tissue appeared macerated.

### Example 6

### Watermelon Tissue DNA Extraction

The same size samples of watermelon leaves and rind tissue are used in DNA extractions. Samples are taken using a sterile scalpel and extracted by a FastDNA kit (Qbiogene, Carlsbad, Calif., USA) according to manufacturer's directions for the extraction of bacterial DNA. Note that part of the FastDNA kit involves the maceration of plant tissue using an apparatus that vigorously shakes a 1.5 ml tube containing the tissue with garnet sand and a ceramic sphere.

### Example 7

### Polymerase Chain Reaction (PCR) Amplification

Polymerase chain reactions are performed with the GeneAmp Kit from Perkin-Elmer (Foster City, Calif.; part no. N808-0009) using 50 mM KCl, 2.5 mM MgCl₂, 10 mM Tris-HCl, pH8.3, and containing 200 μM of each dTTP, dATP, dCTP, and dGTP in 25 μl reactions with 0.05 units/μL of Taq polymerase. Oligonucleotide primers are synthesized by Integrated DNA Technologies (Coralville, Iowa) and are present in reactions at a concentration of 1 pmol/μl each. One microliter containing either 10 ng of purified bacterial genomic DNA or diluted watermelon tissue extract are used as templates. Reactions are run for 35 cycles of 15 s at 94° C., 15 s at 60° C., and 45 s at 72° C. followed by a hold at 72° C. for ten minutes in a Perkin-Elmer Model 9600 or 9700 thermal cycler. Ten microliters of PCR product are loaded on 1.0% agarose gels containing ethidium bromide. Electrophoresis is carried out at 100 V for 45 minutes and products are visualized. Products are compared to a molecular size marker (Phi-X 174 HaeIII digest) and positive controls on the gel to determine that the products scored are the correct size. Results are scored as either positive (+) or negative (−) for the amplification of target DNA. The visible product is considered a positive result if it is the same size as the positive control reaction product and free of non-specific amplification products.

### Example 8

### Design of Species-specific PCR Primers

A sequence was obtained from the GenBank database of the National Center for Biotechnology Information for A. avenae subsp. citrulli 16S rDNA (accession number AF137506, SEQ-ID-NO: 1). In order to exploit the greater diversity found in the adjacent 16S-23S spacer region, the GenBank 16S sequence was investigated for the presence of one of the conserved primers described for amplifying the 16S-23S spacer by Söller et. al. (Int. J. Syst. Evol. Micro. 2000, 50, 909-915.). This revealed the priming site for BITS-1 (SEQ ID NO: 2) described as the conserved region 3 within the rRNA operon (The GenBank sequence for A. avenae subsp. citrulli 16S rDNA shows one difference in the ambiguous base position for the BITS-1 priming site and this is modified to produce a primer labeled Aac-BITS1 (SEQ-ID-NO: 3). The primer sites for the BITS-1 primer and the modified primer for Acidovorax spp. Aac-BITS1 overlap with respect to the known GenBank sequence for the 16S rDNA gene of Acidovorax avenae subsp. citrulli is (figure not shown).

Using these primers as forward primers located in the 16S rDNA along with the reverse primer described by Söller et al (Int. J. Syst. Bacteriol., (2000) 50, 909-915) for the amplification of the Gürtler and Stanisich region 6 priming site located in the 23S rDNA, BITS-2 (SEQ ID NO: 4) the 16S-23S spacer region between the two genes can be amplified. These primers are synthesized and used on all isolates in this study to show positive amplification of bacterial DNA.

A PCR is run using the forward primer Aac-BITS 1 or alternatively BITS-1 with the reverse primer BITS-2 on template DNA from an isolate of Aac (ATCC #29625). The 16S-23S spacer region product obtained is sequenced using the primers with which it was amplified after being purified of free nucleotides and primer dimers with a Qiagen (Valencia, Calif., USA) PCR Product Clean-up kit. This novel sequence for Aac is identified as SEQ ID NO: 5. We identify this sequence as novel because no other Acidovorax spp. or closely related bacterial results are returned when it is compared to the GenBank database.

To design primers for use in the amplification or sequencing of the Aac 16S-23S spacer DNA, we targeted the new sequence 21Aug00F (SEQ ID NO: 5, described above). Forward primer Aac-BITS3 (SEQ ID NO: 6) and reverse primers Aac-BITS4 (SEQ ID NO: 7) and Aac-BITS6 (SEQ ID NO: 8, complement to Aac-BITS4) are designed to prime with the Aac 16S-23S spacer region. Similarly, Aac-BITS5 (SEQ ID NO: 9) is designed to target a region of 21Aug00F that was similar to the rRNA operon conserved region 5 described by Gürtler and Stanisich. Primer Aac-BITS7 (SEQ ID NO: 10) is designed to be used as a forward primer located within the known Aac 16S rDNA sequence. Also of utility is a primer modified from the conserved, reverse priming 16S rDNA primer 1100r (5′GGGTTGCGCTCGTTG-3′) described by D. J. Lane (“16S/23S rRNA sequencing” Nucleic acid techniques in bacterial systematics. Stackebrandt and Goodfellow eds. 1991 John Wiley and Sons, England, p 133) to be used as a forward primer (our 1100F, SEQ ID NO: 11).

These primers are synthesized for use in amplification or sequencing of the Aac rRNA operon including regions of the 16S rDNA and 16S-23S spacer. Using them we obtain sequencing products that include parts of the known 16S rDNA as well as novel sequence material. Sequences pCRAacD2-1 (SEQ ID NO: 12) and pCRAacD4-1 (SEQ ID NO: 13) are obtained by using primers 1100F and Aac-BITS6 in PCR reactions on DNA extracted from Aac isolate ATCC 29625. Sequences pCRAAC29625-C2-17 (SEQ ID NO: 14) and pCRAAC29625-C4-22 (SEQ ID NO: 15) are similarly obtained by using primers Aac-BITS7 and BITS2. The Aac-BITS7 and BITS2 product sequences are compared and with the exception of one mismatch form a consensus sequence we identify as 29625BITS (SEQ ID NO: 16).

When sequence fragments SEQ ID NO: 1, SEQ ID NO: 12 and 13 and SEQ ID NO: 16 are aligned a contiguous sequence is formed showing that the 16S-23S spacer sequences obtained are adjacent to the 16S rDNA sequence from GenBank as predicted and belongs to A. avenae subsp. citrulli as opposed to being a non-specific amplification product. The 16S-23S spacer region is also amplified from a total of four additional isolates of A. avenae subsp. citrulli (isolate identifiers: AAC zucchini #6, AAC yellow squash, AAC IA-canteloupe, and AAC 94-21 as identified in Table 1) using primers AacBITS7 and BITS2. The products are then cloned into a sequencing vector (TOPO-TA vector, Invitrogen Corporation) and sequenced. The sequences obtained are identified as SEQ ID NO: 17, 18, 19, and 20, respectively). Using the same primers 16S-23S spacer region sequences are obtained for Aac isolates 92-17, Au-9, and 98-16 (also identified in Table 1). These PCR products are purified using the Qiagen (Valencia, Calif., U.S.A.) PCR Product Clean-up kit then sequenced using the primers used in their amplification. The 16S-23S spacer region sequences obtained for these isolates are identified as SEQ ID NO: 21, 22, 23, and 24, respectively). The sequences are aligned with the 29625BITS sequence obtained from isolate ATTC 29625 and minimal disagreements are found among them. This alignment is used in the design of primers to ensure that they will cross-react with any isolate of A. avenae subsp. citrulli.

The 29625BITS sequence is then truncated by removing all 16S rDNA sequence from it. The sequence produced is identified as “29625BITS minus 16S” SEQ ID NO: 24. Using the 16S-23S spacer region sequence obtained in a BlastN of the GenBank database the closest published match found is Ralstonia solanacearum (Accession Number AJ277856, SEQ ID NO: 25). Acidovorax and Ralstonia are both genera belonging to the beta subdivision of the proteobacteria. An alignment is made of this sequence with our A. avenae subsp. citrulli spacer sequence. The alignment is analyzed for divergences between the two sequences. The divergences allow the development of primers for the amplification of A. avenae subsp. citrulli but not the closest sequence match in GenBank, R. solanacearum. Five primers are designed to target regions that contain the greatest differences in sequence between the two sequences analyzed. These are composed of four primers designed for use as forward primers, Aac-BITS8, Aac-BITS9, Aac-BITS10, and Aac-BITS11 and one primer designed for use as a reverse primer, Aac-BITS12, (SEQ ID NOs: 26, 27, 28, 29, and 30, respectively).

The primers are then subjected to a BlastN analysis to find other sequences in GenBank with which they would cross-react. There are no significantly similar bacterial sequences to any of the five primers designed. The primers targeting Aac are synthesized.

Additionally, 16S-23S spacer sequences of more closely related species including A. avenae subsp. avenae, A. facilis, and Pseudomonas acidovorans are obtained by PCR using primers Aac-BITS 1 and BITS2, cloning the products, and sequencing them. Sequences are obtained for A. avenae subsp. avenae isolate 78-5 (SEQ ID NO: 31), A. facilis isolate 94-1 (SEQ ID NO: 32), and Pseudomonas acidovorans isolate ATCC 15669 (SEQ ID NO: 33). These are analyzed for the forward priming sites of Aac-BITS8-11. All of the sites are maintained in the Acidovorax avenae subspecies avenae but divergences are found in those sites for A. facilis, and Pseudomonas acidovorans. Thus the primers are not expected to cross-react with these species. Other 16S-23S spacer sequences were obtained for other isolates in a similar manner including A. avenae subsp. citrulli isolate ATCC 33619 (SEQ ID NO: 34), A. avenae subsp. avenae isolate ATCC 19307 (SEQ ID NO: 35), A. avenae subsp. avenae isolate 78-5 (SEQ ID NO: 36), and Delftia acidovorans isolate ATCC 15668 (SEQ ID NO: 37), as well as 16S-23S spacer sequences for other bacteria found on watermelons including Xanthomonas curcurbitae isolate ATCC 23378 (SEQ ID NO: 38) and Erwinia tracheiphila isolate ATCC 27003 (SEQ ID NO: 39). These are obtained by PCR using primers BITS1 and BITS2, cloning the products, and sequencing them. When these are analyzed for the forward priming sites of Aac-BITS8-11 we see that all are maintained in the Acidovorax avenae subspecies avenae and citrulli isolates but divergences are found in the 16S-23S spacers for isolates from Delftia acidovorans, Xanthomonas curcurbitae, and Erwinia tracheiphilia. No cross-reaction is expected among the species whose spacer sequences show divergences with the Aac-BITS8-11 priming sites.

### Example 9

### Determination of Primer Specificity to Purified Genomic DNA

PCRs are performed using different primer combinations (Table 5) in attempt to amplify single specific fragments. PCR reaction mixtures for each of the primer combinations in Table 5 are run against a negative control (no DNA added) and ten-fold dilutions of A. avenae subsp. citrulli genomic DNA ranging from 10 ng to 1 pg per reaction.

TABLE 5
Possible combinations of PCR primers for the specific amplification
of A. avenae subsp. citrulli

| | | Approximate |
|---|---|---|
| | | Product Size |
| 5′ primer | 3′ primer | (bp) |
| AacBITS8 | AacBITS12 | 391¹ |
| AacBITS9 | AacBITS12 | 383² |
| AacBITS10 | AacBITS12 | 386³ |
| AacBITS11 | AacBITS12 | 378⁴ |

¹Amplifies target but with low sensitivity
²Amplifies target but with low sensitivity
³Amplifies target well
⁴Amplifies target but with lower sensitivity than AacBITS10/AacBITS12

Several primer pairs amplify single products from target DNA with all negative controls free of both specific and nonspecific reaction products. The primer pair that results in the best amplification for its specific targets with no cross-amplification was Aac-BITS10/Aac-BITS12. These primers used together in PCR reactions, produce a 386 bp product from the A. avenae subsp. citrulli rDNA spacer region. Additionally, PCR products using primers Aac-BITS10 and Aac-BITS12 were amplified from A. avenae subsp. citrulli as well as A. avenae subsp. avenae, cloned as described above, and sequenced. This produces sequences SEQ ID NO: 40 and 41 for A. a. subsp. citrulli and A. a. subsp. avenae, respectively. When these are compared to the AAC 16S-23S spacer sequence SEQ ID NO: 24 we see only minimal disagreements further confirming that our primers are amplifying from the correct gene region of Acidovorax spp.

### Example 10

### PCR Primers Specific to Acidovorax avenae subsp. citrulli

The Aac-BITS-10/Aac-BITS-12 primer pair is chosen for further characterization and testing. They are run in PCR master mixes against DNAs from a panel of bacterial species (all isolates in Table 1). Results of each of these tests are shown in Table 6. Seven A. avenae subsp. citrulli isolates, three isolates of A. avenae subsp. avenae, two isolates of A. avenae subsp. cattleyae, and 16 other species are used to show that the assays react with multiple isolates of target DNA without cross-reacting with other, closely-related species. The primer pair Aac-BITS-10/Aac-BITS-12 amplifies from A. avenae subsp. citrulli, but also from A. avenae subsp. avenae, and A. avenae subsp. cattleyae.

TABLE 6
Results of the A. avenae subsp. citrulli
PCR assay against bacterial test isolates

| | Isolate | AacBITS10/AacBITS12 |
|---|---|---|
| Species | Designation | PCR Result |
| A. avenae subsp. citrulli | 94-21 | + |
| A. avenae subsp. citrulli | Zucchini | + |
| A. avenae subsp. citrulli | Yellow squash | + |
| A. avenae subsp. citrulli | IA Cantaloupe | + |
| A. avenae subsp. citrulli | 92-17 | + |
| A. avenae subsp. citrulli | Au-9 | + |
| A. avenae subsp. citrulli | 29625 | + |
| A. avenae subsp. avenae | 19307 | + |
| A. avenae subsp. avenae | 78-5 | + |
| A. avenae subsp. cattyleae | 33619 | + |
| A. avenae subsp. cattyleae | 98-1 | + |
| A. avenae subsp. avenae | 78-5 | + |
| A. avenae subsp. konjaci | 33996 | − |
| Delftia acidovorans | 15668 | − |
| Erwinia tracheiphila | 27003 | − |
| Xanthomonas curcurbitae | 23378 | − |
| Agrobacterium radiobacter | K84 | − |
| Agrobacterium radiobacter | 79-1 | − |
| Ralstonia solanacearum | K60 | − |
| Pseudomonas phaseolicola | 00-1 | − |
| Xanthomonas campestris | 00-1 | − |
| pv. campestris | | |
| Pantoea agglomerans | 99-3 | − |
| Burkholderia cepacia | 92-1 | − |
| Pseudomonas viridflava | 00-1 | − |
| Pseudomonus marginata | 83-1 | − |
| Acidovorax facilis | 94-1 | − |
| Xanthomonas campestris | 00-1 | − |
| pv. vesicatoria | | |
| Pseudomonas aeruginosa | 84-1 | − |

Assays using AacBITS-10 and AacBITS-12 for the detection of A. avenae subsp. citrulli amplify DNA only from A. avenae subsp. citrulli, A. avenae subsp. avenae and A. avenae subsp. cattleyae. No other amplification products are seen from DNA from the other bacterial pathogens listed in Table 6.

### Example 11

Detection of A. avenae subsp. citrulli in field samples using the PCR assay A subset of the field samples documented in Table 2 are tested using the A. avenae subsp. citrulli primers. The results are documented in Table 7.

TABLE 7
Results of A. avenae subsp. citrulli
PCR against field-grown watermelon tissues

| Sample | Variety | Visual |
|---|---|---|
| Designation | Assessment | PCR Result |
| G | Carousel Healthy Leaves? | − |
| H | Carousel Diseased Leaves | − |
| L | Carousel Diseased Leaves | − |
| M1 | Carousel Healthy rinds? | + |
| N | Carousel Diseased rinds | +++++ |
| O | Carousel Diseased rinds | − |

These results are preliminary and inconclusive as to the present sensitivity of the PCR assay for A. avenae subsp. citrulli in infected plant tissue. However, these results demonstrate that it is possible to detect A. avenae subsp. citrulli in infected plant material as seen for samples M1 and N. Additional work is done by sequencing the AacBITS10/AacBITS12 PCR product from watermelon sample N (SEQ ID NO: 42). Sequencing confirmed that the product amplified using these primers is the intended A. avenae spacer target sequence and not a nonspecific amplification product.

### Example 12

### Development of Antibodies Specific to Acidovorax avenae subsp. citrulli

Antibodies are prepared in both rabbit and goat. The rabbit is immunized with a bacterial protein extract derived from the extraction of 12 different isolates of Acidovorax avenae subsp. citrulli. The goat is immunized with a protein observed only in extracts of Acidovorax spp. To obtain the protein, A. avenae subsp. citrulli extract is analyzed by electrophoresis under denaturing and reducing conditions in SDS polyacrylamide gels and stained with Coomassie blue according to Laemmli, U.K. (1970) Nature, London 227: 680-685 along with protein extracts from other bacteria listed in Table 1 including: A. avenae subsp. avenae, A. avenae subsp. cattyleae, A. avenae subsp. konjaci, E. cloacae, B. cepacia, B. gladioli pv. gladioli and D. acidovorans. A single-stained protein band of an approximate molecular weight of 160 kDa which is unique to the Acidovorax spp. is cut from the gel and used as the immunogen. N-terminal sequence analysis using an Applied Biosystems (Foster City, Calif.) model 764A protein sequencer of the approximately 160 kDa protein results in a sequence of DVVGAAPLTATNAAAA (SEQ ID NO: 43). Blast analysis (National Center for Biotechnology Information, Bethesda, Maryland) shows an 83% sequence identity to a hypothetical protein s110456 from Synechocystis sp. (strain PCC6803).

### Example 13

### Western Analysis

Bacterial extracts are analyzed by electrophoresis under denaturing and reducing conditions in SDS polyacrylamide gels and stained with Coomassie blue according to Laemmli, U.K. (1970) Nature, London 227: pp 680-685. The separated proteins were electroblotted onto nitrocellulose (Towbin, H. et al. (1979) PNAS 76: 4350-4354) and probed with antisera prepared against the 160 kDa protein. Goat antibody was detected with alkaline phosphatase-labeled donkey anti-goat IgG (Jackson ImmunoResearch Laboratories, Inc.). The blots were developed with BCIP/NBT substrate (Moss, Inc.). The antibody described in example 12 binds to a 160 kDa protein band from A. avenae subsp. citrulli. The antibody also binds to a 16 kDa protein band from B. cepacia. The antibody does not bind to any proteins made from extracts of A. avenae subsp. avenae, A. avenae subsp. cattyleae, A. avenae subsp. konjaci, A. radiobacter, R. solanacearum, P. phaseolicola, X campestris pv. campestris, P. agglomerans, P. viridiflava, P. marginata, A. facilis, X campestris pv. vesicatoria and P. aeruginosa.

### Example 14

### A. avenae subsp. citrulli ELISA

This immunoassay is a semi-quantitative sandwich assay for the detection of Acidovorax avenae subsp. citrulli. It employs two polyclonal antibodies that have been immunoaffinity purified against Acidovorax avenae subsp. citrulli protein extract. First the plates are coated at 4° C. overnight with the rabbit antibody at a concentration of 3 μg/ml, diluted in borate buffered saline pH 8.5. The plates are washed five times with a Tris base buffer pH 8.0 (wash buffer). Note: the same wash step is performed after each incubation period to remove unbound antibodies/samples. Plates are then blocked for 45 min. at room temperature (RT) with PBS/Tween-20/BSA buffer pH 7.4 (diluent). Fifty microliters of each sample are added to the plate and incubated for 1.5 hr. at RT. The goat antibody (diluted to 1 μg/ml in diluent) is then added to the plates and incubated for 1 hr. at 37° C. The detection antibody (alkaline phosphatase-labeled donkey anti-goat diluted to 1 μg/ml in diluent) is then added to the plates and incubated for 1 hr. at 37° C. Substrate (pNPP) is added and allowed to develop for 30 min at RT. The absorbance is then measured at 405 nm with 492 nm as a reference.

### Example 15

### Determination of ELISA Sensitivity

Different concentrations of A. avenae subsp. citrulli are measured in the ELISA to determine the sensitivity. The ELISA is able to detect 5×10⁴ bacteria/ml (data not shown).

### Example 16

### A. avenae subsp. citrulli Immunostrips

The lateral-flow immunostrip consists of a detection membrane of nitrocellulose, supported on a plastic backing, in which a 1 mm line of specific Acidovorax avenae subsp. citrulli antibody is sprayed. A reagent control line of donkey anti-rabbit antibody is sprayed in parallel above the first antibody line. The membrane is flanked on the top by an absorption pad and on the bottom by a pad containing dried colloidal gold-labeled rabbit anti-Acidovorax avenae subsp. citrulli antibody. A sample application pad then flanks the colloidal gold pad. This completed card is then cut into 4 mm test strips to fit into a plastic cassette with an oval sample application well positioned above the sample pad and a rectangular detection window positioned above the detection membrane. The assay is performed by adding 150 μl of extracted tissue to the sample well. After waiting approximately 5-10 minutes, the results appear in the result window. If Acidovorax avenae subsp. citrulli is present in the sample, a double red line appears in the result window. The lower line indicates the presence of Acidovorax avenae subsp. citrulli, while the upper line is the control line signaling a properly working device. If no Acidovorax avenae subsp. citrulli is present, only one single red control line appears in the result window.

### Example 17

### Specificity of the A. avenae subsp. citrulli ELISA and A. avenae subsp. citrulli Immunostrips

The percent cross-reactivity of various indigenous bacterial isolates is determined by spiking each bacterial extract into the ELISA buffer and measuring the resultant A. avenae subsp. citrulli concentration. All bacteria tested are less than 0.2% cross-reactivity in the ELISA with the exception of Acidovorax avenae subsp. avenae which shows a 0.4% cross-reactivity (Table 8).

TABLE 8
Specificity of A. avenae subsp. citrulli ELISA

| | | | Percent |
|---|---|---|---|
| | Concentration | Concentration | Cross |
| Species | Added | Measured | Reactivity |
| Enterobacter cloacae | 2000 ng/ml | 1.53 ng/ml | 0.08% |
| Burkholderia gladioli | 2000 ng/ml | 3.17 ng/ml | 0.16% |
| pv gladioli | | | |
| Delftia acidovorans | 2000 ng/ml | 1.71 ng/ml | 0.09% |
| A. avenae subsp. avenae | 2000 ng/ml | 8.73 ng/ml | 0.44% |
| A. avenae subsp. cattleyae | 2000 ng/ml | 3.09 ng/ml | 0.15% |
| A. avenae subsp. konjaci | 2000 ng/ml | 2.57 ng/ml | 0.13% |
| Xanthomonas cucurbitae | 2000 ng/ml | 0 ng/ml | <0.001% |
| Erwinia tracheiphil | 2000 ng/ml | 0 ng/ml | <0.001% |
| Agrobacterium radiobacter | 1000 ng/ml | 0.64 ng/ml | 0.06% |
| Pantoea agglomerans | 1000 ng/ml | 0.17 ng/ml | 0.02% |
| Ralstonia solanacearum | 1000 ng/ml | 0.01 ng/ml | <0.001% |
| Pseudomonas aeruginosa | 1000 ng/ml | 0.41 ng/ml | 0.04% |
| Burkholderia cepacia | 1000 ng/ml | 0.33 ng/ml | 0.03% |
| Acidovorax facilis | 2000 ng/ml | 5.13 ng/ml | 0.26% |

A. avenae subsp. citrulli immunostrips are also tested for cross-reactivity with 20 indigenous bacteria by spiking bacterial extracts into negative watermelon seedling extract.

The results are recorded using a plus/minus scale. Only Acidovorax avenae subsp. konjaci and Acidovorax avenae subsp. avenae show minimum cross-reactivity with the A. avenae subsp. citrulli immunostrips (Table 9).

TABLE 9
Cross-reactivity of Aac Immunostrips

| | Antigen | |
|---|---|---|
| | Concentration | |
| | Added to Healthy | Immunostrip |
| Species | Seedling Extract | Reactivity |
| Agrobacterium radiobacter | 10 μg/ml | −− |
| Pantoea agglomerans | 10 μg/ml | −− |
| Ralstonia solanacearum | 10 μg/ml | −− |
| Pseudomonas aeruginosa | 10 μg/ml | −− |
| Burkholderia cepacia | 10 μg/ml | −− |
| Xanthomonas cucurbitae | 10 μg/ml | −− |
| A. avenae subsp. konjaci | 10 μg/ml | +/− |
| A. avenae subsp. cattleyae | 10 μg/ml | −− |
| Delftia acidovorans | 10 μg/ml | −− |
| Enterobacter cloacae | 10 μg/ml | −− |
| Erwinia tracheiphil | 10 μg/ml | −− |
| A. avenae subsp. avenae | 10 μg/ml | +/− |
| Burkholderia gladioli pv gladioli | 10 μg/ml | −− |
| Acidovorax facilis | 10 μg/ml | −− |
| A. avenae subsp. citrulli | 1 μg/ml | + |

### Example 18

### Detection of Acidovorax avenae subsp. citrulli from Field Samples Using A. avenae subsp. citrulli Immunostrips

Extracts from 25 watermelon samples (either leaf or fruit) are tested using the A. avenae subsp. citrulli immunostrips. After the samples were extracted, a test immunostrip is placed into each tube and allowed to react for 10 min. The tests are scored visually using a plus and minus scale. The weakest reaction is scored as +/− and the strongest reaction is scored as +++. These results are compared to results obtained using a commercially available Bacterial Fruit Blotch Affitips Kit (Hydros, Inc. Falmouth, Mass.). The extracts are also tested in the A. avenae subsp. citrulli ELISA. The results are summarized in Table 10.

TABLE 10
Results of A. avenae subsp. citrulli Immunostrip Testing
on Field Samples of Watermelon

| | | Immunostrip | Hydros | ELISA |
|---|---|---|---|---|
| Sample | Variety | Result | Result¹ | Result |
| A | Stars & Stripes | − | − | 0.087 |
| B | Stars & Stripes | − | − | 0.133 |
| C | Stars & Stripes | − | − | 0.162 |
| D | Stars & Stripes | +/− | − | 0.091 |
| E | Stars & Stripes | − | − | 0.094 |
| F | Carousel | − | − | 0.112 |
| G | Carousel | −/+ | − | 0.216 |
| H | Carousel | −/+ | − | 0.329 |
| I | Carousel | +/− | − | 0.134 |
| J | Carousel | + | − | 0.334 |
| K | Carousel | + | − | 0.484 |
| L | Carousel | ++ | − | 2.416 |
| M1 | Carousel | + | − | 0.812 |
| M2 | Carousel | +/− | NT | 0.183 |
| N | Carousel | ++ | + | 3.048 |
| O | Carousel | − | − | 0.095 |
| P | Carousel | ++ | + | 2.606 |
| Q | Carousel | ++ | + | 2.524 |
| R | Carousel | ++ | + | 2.409 |
| S | Fandango | −/+ | − | 0.673 |
| T | Fandango | +/− | − | 0.131 |
| U | Fandango | ++ | + | 3.089 |
| V | Fandango | ++ | NT | 2.182 |
| W | Sugartime | +/− | NT | 1.018 |
| X | Sugartime | +/− | NT | 1.434 |
| Y | Sugartime | + | NT | 2.361 |

¹NT = not tested with Hydros Affinitip.

While the present invention has been described with reference to specific embodiments thereof, it will be appreciated that numerous variations, modifications, and further embodiments are possible, and accordingly, all such variations, modifications and embodiments are to be regarded as being within the scope of the present invention.

Numerous patents, applications and references are discussed or cited within this specification, and all are incorporated by reference in their entireties.

What is claimed is:

1. A nucleic acid molecule encoding a 16S-23S spacer DNA sequences for the bacterial species Acidovorax avenae subsp. citrulli, Acidovorax avenae subsp. avenae, Xanthomonas curcurbitae, and Erwinia tracheiphila.

2. The nucleic acid molecule of claim 1, wherein the 16S-23S spacer DNA sequence is SEQ ID NOS: 5, 12-24, 31, 34-36, or 38-42.

3. A nucleic acid molecule having sequence identity with at least 10 contiguous nucleotides of the 16S-23S rDNA spacer sequence from Acidovorax avenae subsp. citrulli.

4. The nucleic acid molecule of claim 3, wherein the 16S-23S rDNA spacer sequence has the sequence of SEQ ID NOS: 5, 12-24, 34, or 40.

5. A nucleic acid molecule comprising a nucleotide sequence of SEQ ID NOs: 2-4,6-11 or 26-30.

6. A pair of oligonucleotide primers wherein at least one primer consists of the nucleotide sequence of SEQ ID NOS: 2-4, 6-11 or 26-30.

7. A pair of oligonucleotide primers comprising Aac-BITS10 (SEQ ID NO: 28) and Aac-BITS12 (SEQ ID NO: 30).

8. A method for the detection of a bacterial pathogen, comprising the steps of:
(a) isolating DNA from a plant tissue infected with a pathogen;
(b) subjecting said DNA to polymerase chain reaction amplification using at least one primer having sequence identity with at least 10 contiguous nucleotides of a 16S-23S rDNA spacer region sequence of a Acidovorax spp.; and
(e) detecting said bacterial pathogen by visualizing the product or products of said polymerase chain reaction amplification.

9. The method of claim 8, wherein the bacterial pathogen is Acidovorax avenae subsp. citrulli.

10. The method of claim 8, wherein the 16S-23S spacer sequences have the nucleotide sequence of SEQ ID NO: 24.

11. The method of claim 8, wherein at least one primer having the nucleotide sequence of SEQ ID NOS: 2-14.

12. A method for the detection of a bacterial pathogen, comprising the steps of:
(a) isolating DNA from a plant tissue infected with a pathogen;
(b) subjecting said DNA to polymerase chain reaction amplification using at least one primer having sequence identity with at least 10 contiguous nucleotides of a 16S-23S rDNA spacer sequence of Acidovorax avenae subsp. citrulli; and
(c) detecting said bacterial pathogen by visualizing the product or products of said polymerase chain reaction amplification.

13. The method of claim 12, wherein the bacterial pathogen is Acidovorax avenae subsp. citrulli.

14. The method of claim 14, wherein at least one primer having the nucleotide sequence of SEQ ID NOS: 2-4, 6-11, or 26-30.

15. The method of claim 12, wherein a pair of oligonucleotide primers consists of SEQ ID NO: 28 and SEQ ID NO: 30.

16. A diagnostic kit used in detecting a bacterial pathogen comprising at least one primer having at least 10 contiguous nucleotides of a 16S and 16S-23S rDNA spacer sequence of Acidovorax avenae subsp. citrulli.

17. The diagnostic kit of claim 16, wherein at least one primer of SEQ ID NOs: 2-4,6-11 and 26-30 for 16S and 16S-23S rDNA spacer derived primers.

18. The diagnostic kit of claim 16, wherein the pair of primers are SEQ ID NO: 28 and SEQ ID NO: 30.

19. A polypeptide comprising the amino acid sequence of DVVGAAPLTATNAAAA (SEQ ID NO: 43).

20. An antibody that reacts with a polypeptide having the N-terminal amino acid sequence of the polypeptide of claim 19.

21. An immunoassay for the detection of Acidovorax avenae subsp. citrulli that uses the antibody of claim 20.

22. The immunoassay of claim 21, wherein the immunoassay is an ELISA or lateral flow strip format.

23. The immunoassay of claim 21, wherein the immunoassay is used to detect the presence of Acidovorax avenae subsp. citrulli in cucurbit hosts.

24. A kit for the detection by the immunoassay of claim 21 comprising a carrier being compartmented to receive in close confinement therein:
(f) a means of extraction of a test substance in the presence of a primary antibody capable of binding to the test substance wherein said primary antibody is conjugated to a means of detection;
(g) solid phase format having a significant measurement in three dimensions to form a substantial volume with a plurality of interstitial spaces capable of capturing a complex formed by the primary antibody and the test substance;
(h) a vessel containing a buffer;
(i) reagents reactive with the means of detection to produce a detectable reaction product; and
(j) a means of dispensing said reagents.
